(19) United States
(12) Patent Application Publication
(10) Pub. No.: US 2021/0204883 A1
(43) Pub. Date: Jul. 8, 2021

(54) Systems and Methods for Artifact Reduction in Recordings of Neural Activity

(71) Applicant: The Regents of the University of California, Oakland, CA (US)

(72) Inventor: Shennan A. Weiss, Philadelphia, PA (US)

(21) Appl. No.: 15/999,643

(22) PCT Filed: Feb. 20, 2017
(86) PCT No.: PCT/US2017/018581

§ 371 Date: Oct. 20, 2018

Related U.S. Application Data
(60) Provisional application No. 62/297,202, filed on Feb. 19, 2016.

(51) Int. Cl. A61B 5/00; A61B 5/374; A61B 5/37; A61B 5/291

(52) U.S. Cl. CPC A61B 5/7207; A61B 5/4094; A61B 5/291; A61B 5/725; A61B 5/37; A61B 5/374

(57) ABSTRACT

Disclosed are systems and methods for reducing artifacts in neural activity readings. Electrical readings may be acquired using EEG electrodes secured to a subject. A high-pass or band-pass filter may be applied to the electrical readings to favor frequencies more associated with neurogenic potentials as opposed to myogenic potentials. Independent component analysis (ICA) may be applied to the filtered electrical readings. Selected components in each iteration may be pruned to produce reduced-artifact electrical readings, and the reduced-artifact electrical readings may be presented for further review and analysis.

### CROSS REFERENCE TO RELATED APPLICATIONS

This application claims the benefit of U.S. Provisional Patent Application No. 62/297,202, filed Feb. 19, 2016, the disclosure of which, as well as the references cited therein, is hereby incorporated by reference.

### STATEMENT REGARDING FEDERALLY SPONSORED RESEARCH OR DEVELOPMENT

This invention was made with Government support under NS033221, NS033310, NS071048, awarded by the National Institutes of Health. The Government has certain rights in the invention.

### FIELD

This document concerns an invention relating generally to reduction of artifacts in recordings of neural activity acquired via, for example, scalp and intracranial electroencephalography (EEG), for enhanced characterization of brain function and diagnosis of brain pathology.

### BACKGROUND

Measurements of electrical signals in the brain captured via such methods as functional magnetic resonance imaging (fMRI), magnetoencephalography (MEG), and electroencephalography (EEG), can provide useful information on brain structure and function. Recording and monitoring neural activity has been found to be useful in, for example, characterizing and diagnosing sleep disorders, coma, epilepsy, and other conditions. The scalp EEG, for example, is a critical diagnostic tool for neurologists because of its superb temporal resolution, low cost, and diagnostic utility. However, because both the brain and muscles generate electrical signals, recordings of neural activity may be contaminated by electrical activity of muscles. Such contamination introduce artifacts that make electrical recordings more difficult to accurately and effectively interpret upon visual inspection, or using computerized quantification and analysis tools.

The scalp EEG, for example, is a useful diagnostic tool in the evaluation of seizures, but artifacts introduced by muscle contractions can limit its usefulness because cerebrally-generated potentials may be obscured by myogenic potentials. If the origin and spread of seizures in the brain is not discernable using scalp EEG, additional testing may be necessary, such as positron emission tomography (PET), MEG, ictal single-photon emission computed tomography (SPECT), and intracranial EEG. Such added testing requires more time, increases costs, and exposes patients to additional risks associated with the other procedures and examinations. Often when the seizure onset zone cannot be localized due to muscle contamination it necessitates a repeat admission to the epilepsy monitoring unit, or expensive neuroimaging with MEG or SISCOM SPECT.

Digital filters have been used to increase the likelihood of identifying seizure-onset zones in EEGs with muscle artifacts. Because certain frequencies might be more often associated with electrical activity in muscles than a neural activity of interest, conventional filtering has sought to reduce muscle artifact by attenuating all frequencies beyond a selected value. However, this approach may impair the integrity of the EEG recording because brain-generated potentials can occur in overlapping frequency bands. Others have sought to reduce muscle artifact using independent component analysis (ICA), which is intended to remove artifacts based on source-related features instead of frequencies. However, their analyses have not been as effective as desired, and may introduce confounding artifacts that may lead to erroneous interpretations.

### SUMMARY OF THE DISCLOSURE

Disclosed are systems and methods for reducing artifacts in neural activity readings. In exemplary configurations, electrical readings may be acquired using EEG electrodes secured to a subject. A high-pass or band-pass filter may be applied to the electrical readings to favor frequencies above a high-pass frequency threshold, or within a desired range. In consecutive iterations, independent component analysis (ICA) may be applied to the filtered electrical readings. Selected components in each iteration may be pruned to reduce artifact. Once reduced-artifact electrical readings are produced, they may be displayed for visual inspection, the level of artifact quantified, and/or the artifact reduction process otherwise reported on. With scalp EEGs, a low-pass filter may also be applied to the raw electrical readings, and the low-pass filtered readings reconstituted with the high-pass/band-pass filtered and pruned electrical readings. With intracranial EEGs, a first independent component (IC1) in each consecutive iteration may be pruned. In certain preferred implementations, it may also be determined which EEG electrodes are faulty or otherwise not recording with the same or similar fidelity as other recording electrodes. Such a determination may be made via, for example, mutual information (MI) calculations, and the data from such electrodes (channels) excluded or otherwise deemphasized.

Exemplary versions could be implemented in hardware and/or software for use in, for example, clinical decision-making, or for guiding medical interventions such as epilepsy surgery. The processes for minimizing and rejecting artifact in scalp EEG and intracranial EEG further discussed below can be integrated into both hardware and software for interpreting EEG both visually and quantitatively. The system could also be incorporated in implantable devices for diagnostic monitoring, or as part of an intervention such as closed loop neuro-stimulation. Exemplary implementations can offer an automated and unsupervised reduction of artifacts in scalp and intracranial EEG recordings. Further advantages and features of the invention will be apparent from the remainder of this document in conjunction with the associated drawings.

### DESCRIPTION OF THE DRAWINGS

FIG. 1 depicts an exemplary process of reducing artifacts in EEG readings corresponding with neural activity.

FIG. 2 depicts an alternative exemplary method of processing EEG electrical readings to reduce artifacts therein.

FIGS. 3A-3F illustrate improvement in establishing the seizure focus using an exemplary artifact reduction methodology. The exemplary methodology automatically separates independent components containing myogenic potential from neurogenic potentials in the beta and gamma band on the basis of spatial topography and explained variance.

FIG. 3A shows unprocessed scalp ictal EEG recording that were deemed uninterpretable.

FIG. 3B sows the epoch of FIG. 3A after applying a low pass (<16 Hz) filter demonstrating a lack of a convincing ictal rhythm.

FIG. 3C shows the ictal epoch of FIGS. 3A and 3B after applying a high pass (>16 Hz) filter demonstrating dense muscle artifact.

FIG. 3D provides an example of a mutual information adjacency matrix calculated during an epoch of artifact in the high pass (>16 Hz) filtered scalp EEG recording.

FIG. 3E provides an inverse weight matrix of all independent components across scalp electrode recordings for the seizure represented in the panel of FIG. 3A.

FIG. 3F provides normalized inverse weight matrix of all independent components across scalp electrode recordings for the seizure represented in the panel of FIG. 3A.

FIGS. 4A-4C illustrate that reconstitution of a low pass (<16 Hz) filtered ictal scalp EEG with the high pass (>16 Hz) neurogenic independent components reveals a clear ictal onset.

FIG. 4A provides tentative neurogenic independent components (A1) and myogenic independent components (A2) derived from Infomax independent component analysis (ICA) processing of the high pass (>16 Hz) filtered ictal scalp EEG recording.

FIG. 4B provides the low pass filtered ictal scalp EEG suggesting a possible left frontal onset.

FIG. 4C shows that reconstitution of the low pass filtered EEG recordings with the neurogenic high pass (>16 Hz) independent components results in an ictal EEG that demonstrates a more convincing left frontal onset consisting of beta-gamma oscillations with some clear phase reversals in F3 and F7.

FIG. 5 shows phase amplitude coupling of ripples with inter-ictal discharges or theta oscillations in the seizure onset zone.

FIG. 6 shows ripple amplitude is coupled with inter-ictal discharge or theta oscillation phase over extended recording durations.

FIG. 7 illustrates that phase locked ripple phasor rates can unambiguously lateralize the epileptogenic mesial temporal lobe.

FIG. 8 illustrates that coupling between low frequency iEEG phase and ripple amplitude can be used to identify epileptogenic brain regions.

FIG. 9 depicts a mutual information adjacency matrix of the high frequency oscillation (HFO) (80-600) band pass filtered intracranial EEG calculated across all macroelectrode contacts during epochs of artifact.

FIG. 10 provides results of an exemplary method applied to an intracranial EEG recording (panels A1, A2).

FIG. 11 shows that an exemplary artifact reduction process improves visual inspection of intracranial EEG.

FIG. 12 provides an illustration of the utilization of the artifact index for the differentiation of brain HFOs from artifactual signal.

FIG. 13 demonstrates that application of exemplary methods to reduce and annotate artifact improves the precision of automated and unsupervised HFO detection.

FIG. 13 illustrates that pruning independent component 1 and utilizing the artifact index during HFO detection for artifact differentiation improves the precision of event detection for delineating the seizure onset zone.

FIG. 14 illustrates that exemplary implementations improve the clarity of visual inspection and interpretation of traditional scalp EEG.

FIG. 15 provides an illustration of an exemplary utilization of ICA for artifact reduction, rejection of artifactual ripples, and improvement in ripple signal to noise.

FIG. 16 depicts an exemplary system that could be used to implement processes and approaches disclosed herein, with an EEG device in communication with a computing device.

### DETAILED DESCRIPTION

Exemplary versions of the invention involve transforming electrical recordings from the scalp and brain by reducing muscle contamination, thereby increasing the ratio of signals generated by the brain (i.e., neurogenic potentials) relative to signals generated by muscle (i.e., myogenic potentials). Accurately removing muscle contamination involves reliably producing signals that are exclusively or substantially due to brain or muscle activity, and identifying which signals are of brain origin and which are contaminant. In certain configurations, independent component analysis (ICA), such as Infomax, may be used. It may be assumed that more variance across channels in the high pass (>16 Hz) filtered multichannel scalp recordings, or band pass (80-600 Hz) filtered multichannel intracranial recordings, is accounted for by muscle artifact, or referential signal contamination, than brain potentials. The performance of the ICA to separate muscle potentials from brain potentials may be improved by determining if the mutual information between the filtered signals recorded by each of the electrodes was sufficiently large during epochs containing myogenic potentials, and excluding signals from electrode contacts with insufficient mutual information from the ICA.

In the case of intracranial EEG recordings a bipolar montage that involves digitally subtracting the EEG signals from spatial adjacent electrodes is the traditional methodology for reducing artifact from muscle and the reference electrode signal. A disadvantage of the bipolar montage methodology is that reduces the spatial resolution of intracranial EEG, alters the morphology of the brain generated waveforms, and can potentially reduce the detection of brief events called high-frequency oscillations due to cancellation. In contrast, intracranial EEG recordings can be viewed and analyzed in referential montage i.e. unipolar recordings after utilizing the invention.

For recordings from scalp electrodes, the spatial topology of each resulting independent component may serve as the basis for distinguishing the components containing myogenic potentials from those components containing neurogenic potentials (>16 Hz). The components containing myogenic potentials can be pruned, and the resulting time series reconstituted with the low pass (<16 Hz) filtered signal to reduce the myogenic potentials. For recordings from intracranial electrodes the first independent component typically contained the myogenic potentials, and contaminated reference signal. Pruning this component can dramatically reduce the myogenic potentials in the recordings, and improve the signal to noise ratio for detecting high-frequency brain potentials. Furthermore, a derivation of the first independent component time series in each of the recording contacts can be utilized to demarcate the residual increases in amplitude in the band pass filtered signal generated by muscle, or the contaminated referential signal, for the purpose of excluding artifactual increases in high-frequency energy that can mimic neuron generated high-frequency oscillations, or action potentials.

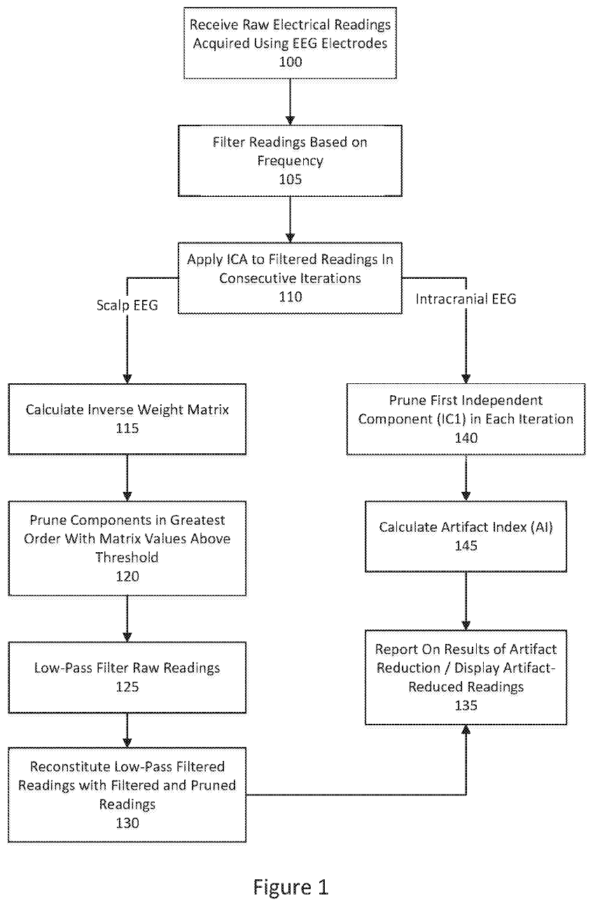

Referring to the exemplary processes depicted in FIG. 1, raw electrical readings corresponding with neural activity may be acquired (100) directly from an EEG device, or indirectly from a computing device storing readings that are to be processed. The electrical readings may be filtered based on frequency (105), such as by being high-pass filtered to favor frequencies above 16 Hz using, for example, a 1000th order symmetric digital finite impulse response (FIR) filter. The filtered readings may then be processed using ICA in consecutive iterations (110), such as Infomax. For scalp EEGs, an inverse weight matrix may be calculated for the components (115), and the components with matrix values above a threshold may be pruned (120). The raw readings may then be low-pass filtered (125) using, for example, a 1000th order symmetric digital FIR filter to favor frequencies below 16 Hz. The low-pass filtered and filtered-and-pruned readings may then be reconstituted (130), and a report produced reporting on artifact reduction analysis and/or visually displaying artifact-reduced EEG readings for inspection, analysis, diagnosis, etc. (135). For intracranial EEGs, the first independent in each iteration may be pruned (140), and the artifact index (AI) calculated, as further discussed.

In certain configurations, EEG scalp recordings may be received and band pass filtered to favor a frequency band of, for example, 16-70 Hz, using a filter, such as a 500th order FIR filter. A power spectral density algorithm may be applied to find extended intervals of elevated high frequency power across electrodes. Certain implementations may then involve calculating the normalized mutual information (MI) adjacency matrix across all scalp electrode contacts during the (16-70 Hz) band-pass filtered artifact epoch of greatest duration. Each scalp EEG electrode may be assigned a single MI value derived from the maximum pairwise MI values in the adjacency matrix. It can then be determined whether this maximum mutual information value exceeded a threshold value, and if that electrode should be included in subsequent artifact reduction processing. If the recording lacks an artifact epoch, or all channels are excluded, artifact reduction may be applied to the referential recordings from all recording electrodes.

The high pass filtered (>16 Hz) scalp EEG may then be separated into consecutive 120-second trials, and each trial processed using an ICA algorithm such as Infomax or CUDAICA. The ICA can separate the (>16 Hz) seizure, or other brain activity, from the (>16 Hz) muscle artifact. The 16 Hz cut-off for the filter may be chosen to isolate the vast majority of the muscle artifact. Independent components that explain an amount of variance above a particular threshold may be excluded from the signal. This high-pass frequency threshold may be selected on the basis of the values of the raw and normalized mixing matrix (i.e., inverse weight matrix) calculated in each of the ICA iterations. It may be assumed in various implementations that the last myogenic component and first neurogenic component can be differentiated on the basis of the inverse weight matrix, which provides the spatial distribution of each component, and identifying the independent component of greatest order with a focal spatial topography defined on the basis of exceeding a normalized threshold in at least one electrode of the inverse weight matrix.

The pruned EEG calculated for each 120-600 second trial of EEG (i.e., each iteration of the ICA) may be concatenated. The entire raw ictal EEG may then be low pass filtered (<16 Hz) using, for example, a 500th order symmetric digital FIR filter. The resulting low pass filtered EEG may be reconstituted with the high pass (>16 Hz) filtered EEG, following the exclusion of the independent components suspected to represent muscle artifact. The reconstituted and modified ictal EEG may be exported (to, e.g., EDF) for subsequent visual inspection and analysis.

In other implementations, the first step may be to determine the EEG electrodes that are not recording brain activity with a fidelity similar to the fidelity of other recording electrodes. The electrode selection process may begin by, for example, by digitally filtering EEG signals above 16 Hz using a 1000th order symmetric digital FIR filter. An epoch of artifact in the EEG band pass filtered record may be determined using normalized mean amplitude across channels threshold based criteria. Next, the mutual information association matrix across electrodes during this epoch of band pass filtered EEG artifact may be calculated. The faulty electrodes that have mutual information values less than a defined threshold may be identified. And these faulty electrodes may be removed from the data and not subject to further processing.

Subsequently, the high pass (>16 Hz) filtered EEG may be processed in consecutive bins/iterations/blocks using ICA (such as Infomax) operating on a paired CPU (central processing unit) and GPU (graphics processing unit) or any other processing configuration deemed suitable. The selection of the independent components to be pruned from the band pass filtered EEG in each bin may be accomplished based on the application of a threshold to the raw and normalized mixing matrix (i.e., inverse weight matrix) calculated in each of the ICA iterations. The independent component of greatest order with raw and normalized mixing matrix values that exceeds the threshold may be pruned from the band pass filtered EEG, along with the independent components of a lesser order. The pruned EEG calculated for each bin (e.g., iteration of Infomax ICA) may be concatenated. Next, the entire unprocessed EEG may be low pass filtered below 16 Hz using the 1000 order symmetric digital FIR filter, and the resulting low pass filtered EEG reconstituted with the >16 Hz high pass filtered and pruned EEG.

In alternative configurations, removal of artifact from intracranial EEG utilizes a similar method with the following exceptions. The process for identifying faulty electrodes may be implemented identically with the exception that the EEG is filtered above 80 Hz (instead of 16 Hz) using a 1000 order symmetric digital FIR filter. The ICA (e.g., Infomax) would operate to process the >80 Hz band pass filtered EEG. Also, only the first independent component may be pruned from the band pass filtered intracranial EEG. Further, the intracranial EEG would not be reconstituted.

If an ictal or inter-ictal high frequency detector is being used, an artifact index a.i.(t) may be calculated using Eqn. 1:

$$AI ( t ) = \frac{\vert {hfo}_{i} ( t ) - ic {1}_{i} ( t ) \vert}{Σ \vert {hfo}_{i} ( t ) \vert \text{/} T} * \frac{1}{I} {\sum }_{i}^{I} \frac{\vert {hfo}_{i} ( t ) - ic {1}_{i} ( t ) \vert}{Σ \vert {hfo}_{i} ( t ) \vert \text{/} T} ; \qquad ( 1 )$$

where hfoᵢ(t) is the band pass filtered EEG or LFP recording (i), and iclᵢ(t) is the band pass filtered EEG or LFP recording (i) after pruning the first independent component. High frequency oscillations (HFOs) detected during an interval when the artifact index exceeds a determined threshold may be deemed artifactual and not tallied.

In other configurations, Infomax ICA may be used with the assumption that more variance across channels in the high pass (e.g., >16 Hz) filtered multichannel scalp recordings, or band pass (e.g., 80-600 Hz) filtered multichannel intracranial recordings, is accounted for by muscle artifact than brain potentials. The performance of the ICA to separate muscle potentials from brain potentials can be improved by determining if the mutual information between the filtered signals recorded by each of the electrodes was sufficiently large during epochs containing myogenic potentials, and excluding signals from electrode contacts with insufficient mutual information from the ICA.

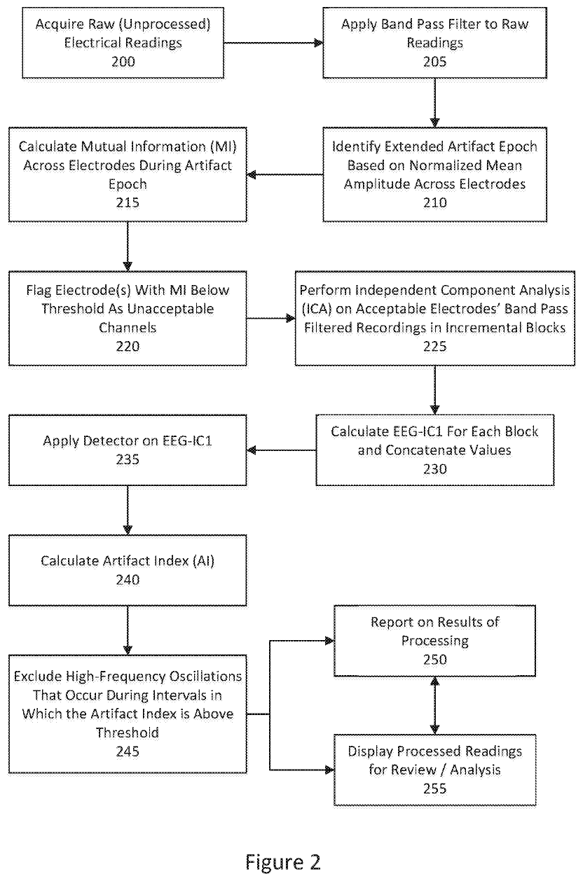

In the implementation represented in FIG. 2, raw electrical readings corresponding with neural activity may be acquired (200) directly from an EEG device, or indirectly from a computing device with readings that are to be processed. A band pass filter may be applied to the raw readings (205) first. An extended artifact epoch based on normalized mean amplitude across electrodes may then be identified (210). Mutual information values may be calculated across electrodes during the artifact epoch (215). Electrodes with MIs below a threshold may be deemed unacceptable and excluded (220). ICA may be performed on the readings acquired using the band-pass filtered readings of the remaining (acceptable) electrodes (225). The first component (IC1) for each block may be calculated, and the values concatenated (230). An ictal or inter-ictal high frequency detector may be applied to the first components (235), and an AI calculated (240). During intervals in which the AI is above a threshold, HFOs may be excluded (245). A report may then generated reporting, for example, the electrodes excluded, the intervals with AIs above threshold, or other quantifications or representations of the analysis (250). Additionally, the processed readings may be displayed for visual inspection and evaluation (255) by a healthcare provider or researcher.

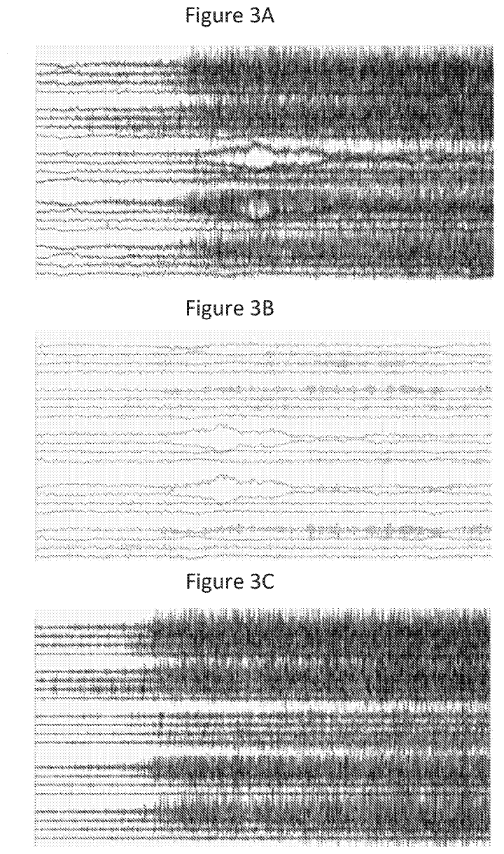

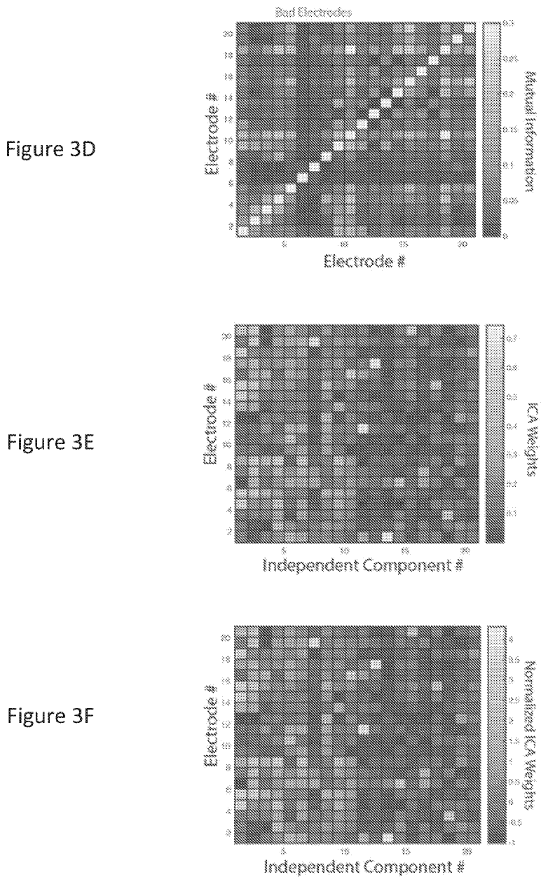

Referring to FIGS. 3A-F, an exemplary artifact reduction method was applied to 23 seizures from eight patients with suspected focal epilepsy undergoing evaluation at the University of California Los Angeles (UCLA) epilepsy-monitoring unit (EMU). The patients and seizures were selected on the basis of a review of consecutive clinical neurophysiology conference presentations in which consensus was reached that the ictal EEG records were uninterpretable due to muscle artifact contamination. For each of these patients, between 1 and 4 uninterpretable seizures were selected for inclusion on the basis of recording quality. Clinical data regarding seizure semiology, inter-ictal activity, and radiological findings were obtained from review of the consensus decisions during clinical neurophysiology conference. The EEGs were acquired using an EEG-1200 amplifier (Nihon-Kohden, Irvine, Calif.) at a sampling rate of 200 Hz. Electrodes were placed according to the 10-20 system, with the additional anterotemporal electrodes in T½. The cheeks were used as a reference. The duration of the exported EEG recording was variable and included the entire seizure and extended peri-ictal epoch.

The process separated the high-pass filtered (>16 Hz) scalp EEG recordings into putative neurogenic and myogenic components. Specifically, the process automatically separated independent components containing myogenic from neurogenic potentials in the beta and gamma band on the basis of spatial topography and explained variance. FIG. 3A shows an unprocessed scalp ictal EEG recording deemed uninterpretable as a result of artifactual contamination. FIG. 3B shows the same epoch after application of a low pass (<16 Hz) filter demonstrating a lack of a convincing ictal rhythm. FIG. 3C shows the ictal epoch after application of a high pass (>16 Hz) filter demonstrating dense muscle artifact.

FIG. 3D provides an example of a mutual information adjacency matrix calculated during an epoch of artifact in the high pass (>16 Hz) filtered scalp EEG recording. Three scalp electrode recordings exhibited relatively low mutual information with all other electrodes and were designated poor quality and excluded from further processing to optimize Infomax ICA based artifact reduction. The inverse weight matrix is shown in FIG. 3E, and the normalized inverse weight matrix in FIG. 3F, for all independent components across scalp electrode recordings for the seizure in FIG. 3A. Independent components 1-13 exhibited strong focality and were designated as containing myogenic potentials, while independent components 14 and above were designated neurogenic.

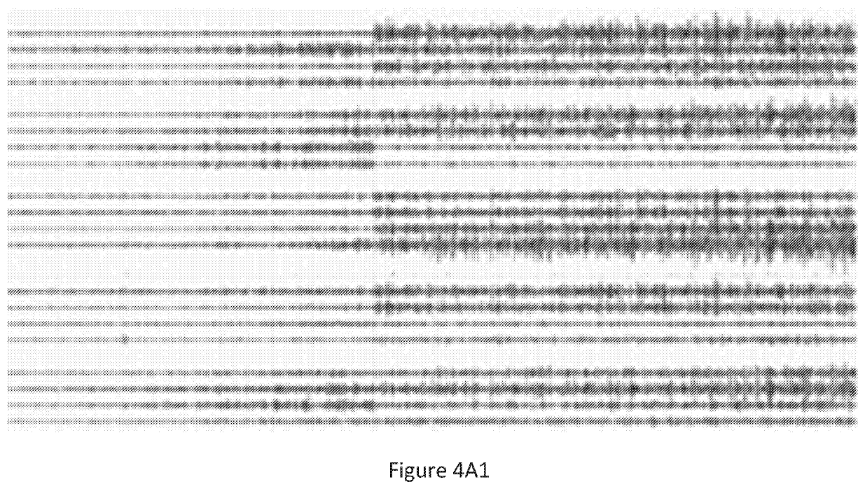

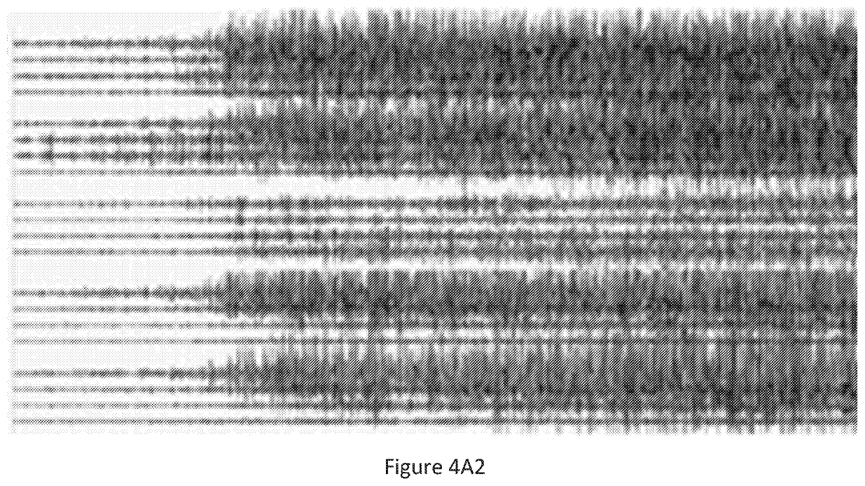

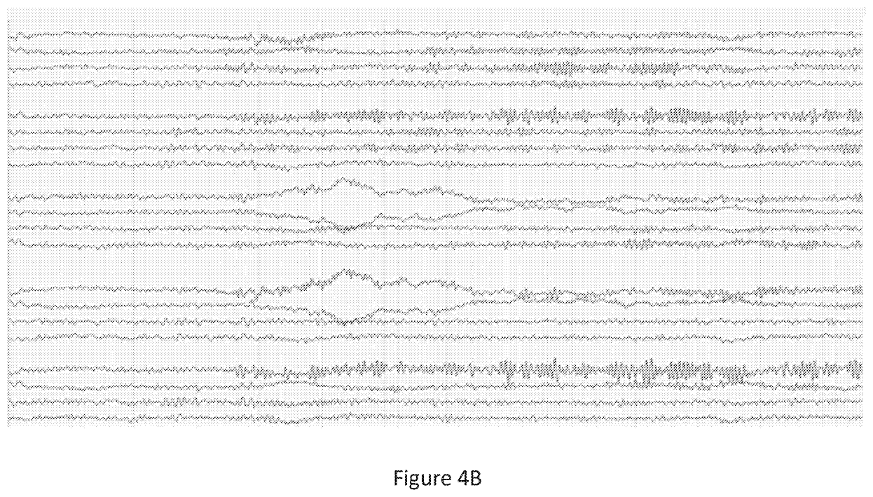

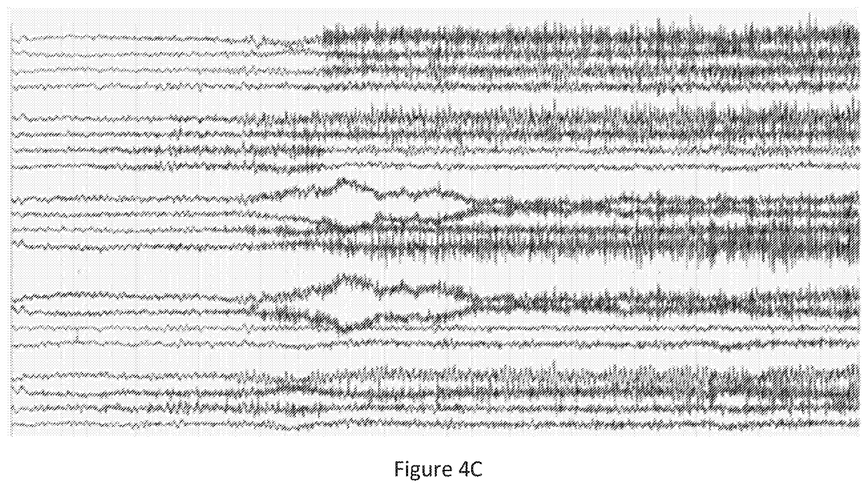

Referring to FIGS. 4A-C, after pruning the putative myogenic components, the putative neurogenic components may be reconstituted with the low-pass filtered (<16 Hz) scalp EEG. Ictal onset is revealed with reconstitution of the low pass (<16 Hz) ictal scalp EEG with the high pass (>16 Hz) neurogenic independent components. In FIG. 4A, the tentative neurogenic independent components (A1) and myogenic independent components (A2) derived from Infomax ICA processing of the high pass (>16 Hz) filtered ictal scalp EEG recording. The largest amplitude activity in the neurogenic components are evident frontally and in the left hemisphere. In FIG. 4B, the low pass filtered ictal scalp EEG suggests a possible left frontal onset but a convincing ictal rhythm is lacking. In FIG. 4C, reconstitution of the low pass EEG with the neurogenic high pass (>16 Hz) independent components results in an ictal EEG that demonstrates a more convincing left frontal onset consisting of beta-gamma oscillations with some clear phase reversals in F3 and F7.

As suggested above, an accurate method to remove EMG artifact ideally depends on (1) the ability of the method to reliably produce signals that are, exclusively or mainly, EEG or EMG, and (2) the ability to identify which signals are of brain origin and which are contaminant. Scalp EEG electrodes record weighted and summated far-field signals from all brain and muscle sources, as well as near-field electrode noise generated at the electrode/skin interface. The decomposition of scalp EEG data into components with maximally independent time courses using independent component analysis results in time series that may resemble single equivalent dipoles because of the bias towards increased local connectivity in neurons and myocytes as compared to long distance connectivity.

Exemplary versions of the invention are unique in that ICA is utilized for the exclusive purpose of identifying components that are generated by scalp myogenic potentials from components generated by cortical neurogenic potentials above the alpha band (see FIGS. 3A-F). Exemplary implementations may be based on three assumptions: 1) that more variance in the (>16 Hz) high pass filtered EEG across electrodes is accounted for by scalp myogenic potentials than cortical neurogenic potentials (FIGS. 3E, 3F); 2) that components containing scalp myogenic potentials will have a spatial topography (FIGS. 3E, 3F) that is more focal than components with cortical neurogenic potentials; and 3) the performance and reliability of ICA to separate scalp myogenic and neurogenic potentials will improve if the mutual information between all the (>16 Hz) high pass filtered EEG signals is sufficiently large during artifactual epochs (see, e.g., FIG. 3D).

Using ICA algorithms in which a decreasing order of the EEG variance is accounted for by each additional independent component, such as Infomax ICA to the high pass filtered (>16 Hz) scalp EEG, will result in an array of independent components ordered 1 through N (FIGS. 3E, 3F). The independent components of a lower order account for scalp myogenic potentials, while the higher order components account for neurogenic cortical potentials. Based on the second assumption, the last myogenic component and first neurogenic component can be differentiated on the basis of the inverse weight matrix (FIG. 3E, 3F) that provides the spatial distribution of each component, and identifying the independent component of greatest order with a focal spatial topography defined on the basis of exceeding a normalized threshold in at least one electrode of the inverse weight matrix.

Hence, by pruning this highest order component containing myogenic potentials and all independent components of a lower order, that also contain myogenic potentials, from the high pass filtered EEG (>16 Hz) (FIG. 4A, A2 on the right) the myogenic potentials in the high pass (>16 Hz) filtered EEG are reduced, whereas the independent components accounting for neurogenic cortical activity remain. Thus, after pruning the ratio of neurogenic activity to myogenic activity in the high pass (>16 Hz) filtered EEG increases relative to the original signal (FIG. 4A).

An EEG recording with a full frequency content, but with reduced scalp myogenic artifact, can then be reconstituted (FIG. 4C) by combining the low pass (<16 Hz) filtered EEG (FIG. 4B) with the high pass (>16 Hz) EEG that has been pruned of myogenic activity (FIG. 4A, A1 on the left).

Computations for the analysis corresponding to FIGS. 3A-F and 4A-C were carried out using compiled Matlab 8.4 (Mathworks) custom scripts on a cluster of HP SL230s Gen 8 ES-2670 nodes with dual-eight-core 2.6 GHz Intel ES-2670 central processing units, 4 GB of memory per core, and NVIDIA Tesla graphics processing units. Ictal and inter-ictal scalp recordings up to an hour duration were imported as EDF files. All channels of the scalp EEG were band pass filtered (16-70 Hz) using a 500th order FIR filter, and a simple algorithm designed to find extended intervals of elevated high frequency amplitude across electrodes was used to select for artifacts in the recording. The normalized mutual information adjacency matrix was calculated across all scalp electrode contacts during the (16-70 Hz) band pass filtered artifact epoch of greatest duration. Each scalp electrode was assigned a single MI value of its maximum pairwise MI values in the adjacency matrix. If this mutual information value did not exceed a threshold its respective electrode was deemed a poor recording contact and was excluded from further analysis. In the case that the recording lacked artifact, or all channels were deemed poor recording contacts, all channels were included in the analysis.

The high pass filtered (>16 Hz) intracranial EEG was then binned into 120 second trials and each trial was processed using CUDAICA, an Infomax ICA algorithm that operates on a paired CPU and GPU and significantly reduces processing time. The selection of the independent components to be pruned from the (>16 Hz) band pass filtered EEG in each bin is calculated on the basis of applying a threshold to the raw and normalized mixing matrix (i.e., inverse weight matrix) calculated in each of the ICA iterations. The independent component of greatest order with raw and normalized mixing matrix values that exceeds the threshold is pruned from the band pass filtered EEG along with the independent components of a lesser order. The pruned EEG calculated for each bin (i.e., iteration of CUDAICA) was concatenated. Subsequently the entire raw EEG was low pass filtered (<16 Hz) using a 500th order symmetric digital FIR filter. The resulting low pass filtered EEG was reconstituted with the high pass (>16 Hz) filtered and pruned EEG containing the neurogenic independent components. The complete analysis of a scalp EEG recording was performed in less than the recording time.

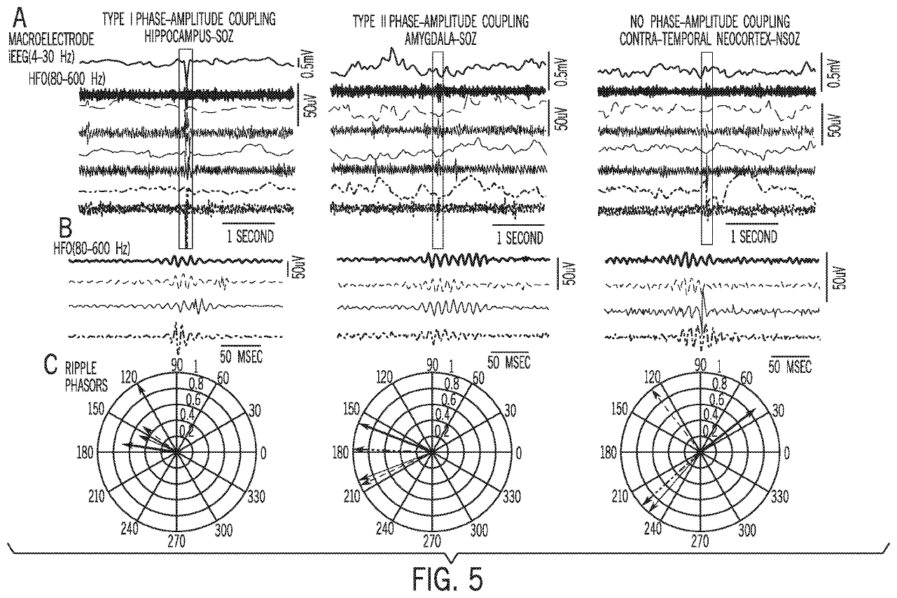

Other exemplary versions will now be discussed in the context of phase amplitude coupled ripples in the intracranial EEG as a precise biomarker of the seizure onset zone in mesial temporal lobe epilepsy. Referring to FIG. 5, ripples are phase amplitude coupled with both inter-ictal discharges or theta oscillations in the seizure onset zone. Panel A provides four examples of the low frequency (4-30 Hz, top) and corresponding high frequency (80-150 Hz, bottom) iEEG recorded from the hippocampal (left) and amygdala (center) mesial contacts located in the seizure onset zone, as well as a neocortical temporal contact (right) located contralateral to the seizure onset zone. The traces are aligned to ripple events (grey bar). The ripples recorded from the hippocampus and amygdala are generated during inter-ictal epileptiform discharges, and the trough of theta oscillations, respectively. Panel B illustrates expansion of the aligned HFOs illustrated by the grey bar in panel A. Panel C provides a polar plot of the corresponding four ripple phasors for each case demonstrating phase locking exclusively in the seizure onset zone.

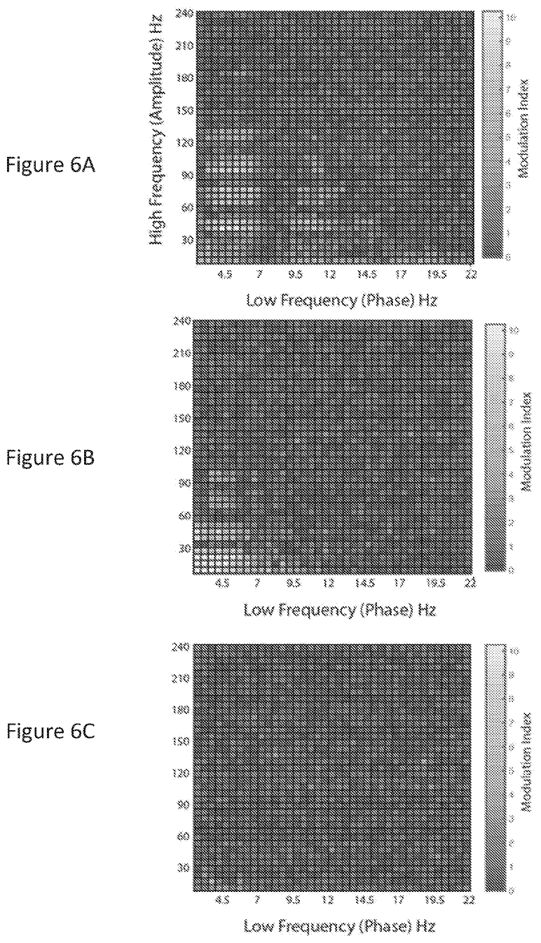

Illustrated in FIG. 6, ripple amplitude is coupled with inter-ictal discharge or theta oscillation phase over extended recording durations. The modulation index is calculated on the basis of the analytic amplitude (5-240 Hz) and analytic phase (2-22 Hz) of one hour recordings from the three intracranial electrodes shown in FIG. 5. Coupling between theta/alpha band activity phase and gamma/ripple band amplitude is evident in the epileptogenic hippocampus, whereas coupling between theta band activity and gamma/ripple band amplitude is evident in the epileptogenic amygdala. In the non-epileptogenic neocortex, that is contralateral to the seizure onset zone, no phase amplitude coupling is evident.

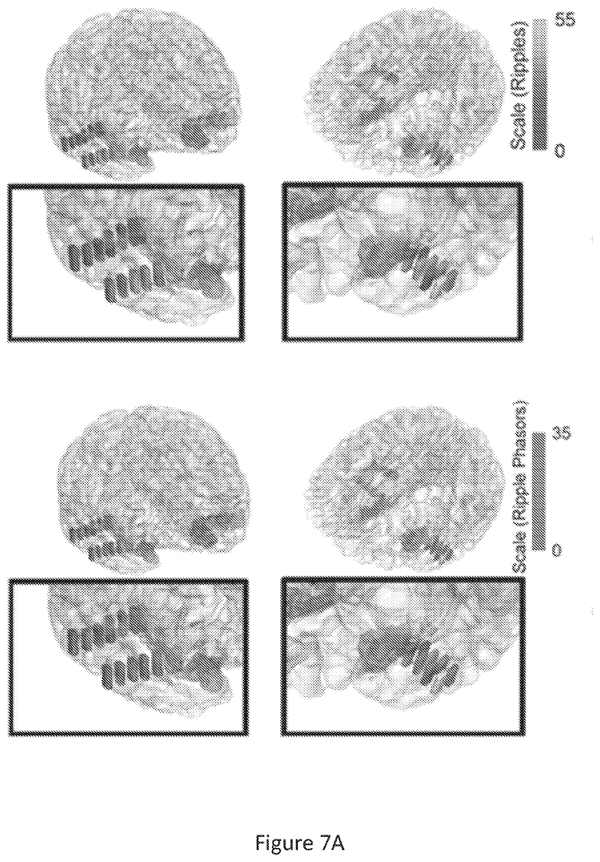

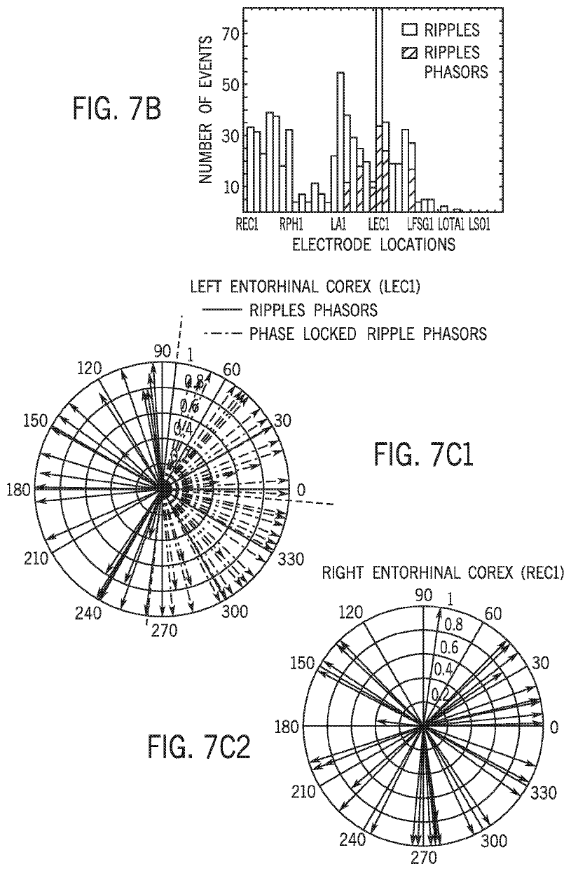

Referring to FIG. 7, phase locked ripple phasor rates can unambiguously lateralize the epileptogenic mesial temporal lobe. Panels A1 and A2 correspond with coregistered CT-MRI of patient number 448 with a left mesial temporal seizure onset zone, with the ripple rates shown in A1, and the phase locked ripple phasor rates shown in panel A2. Panel B provides a bar plot of the number of ripples and phase locked ripple phasors. It is noted that the phase locked ripple phasor rates unambiguously lateralized the seizure onset zone. In panel C, polar plots of the ripple phasors and phase locked ripple phasors for the left (C1) and right (C2) entorhinal cortex. The dashed line represents the mean phase angle of the phase locked ripple phasors, dashed black line represents the division between phase locked and non-phase locked ripple phasors.

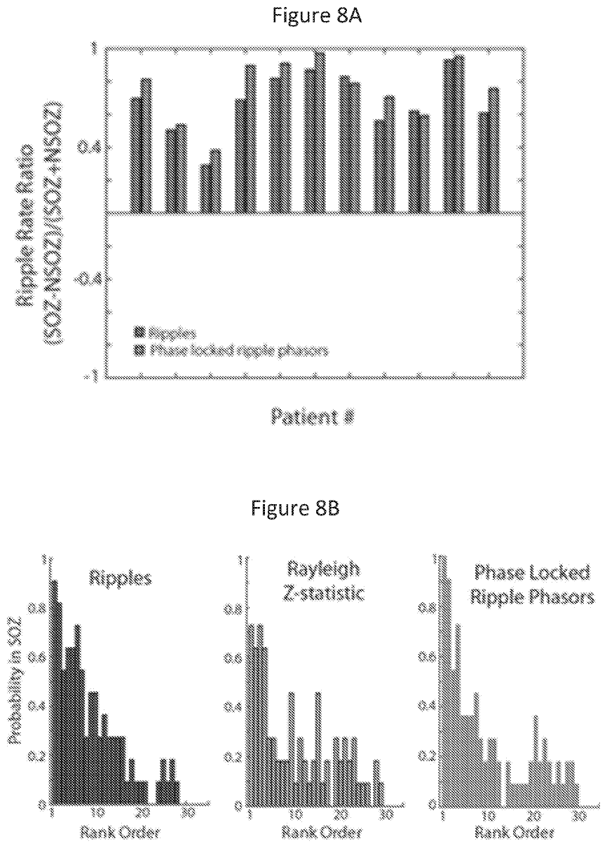

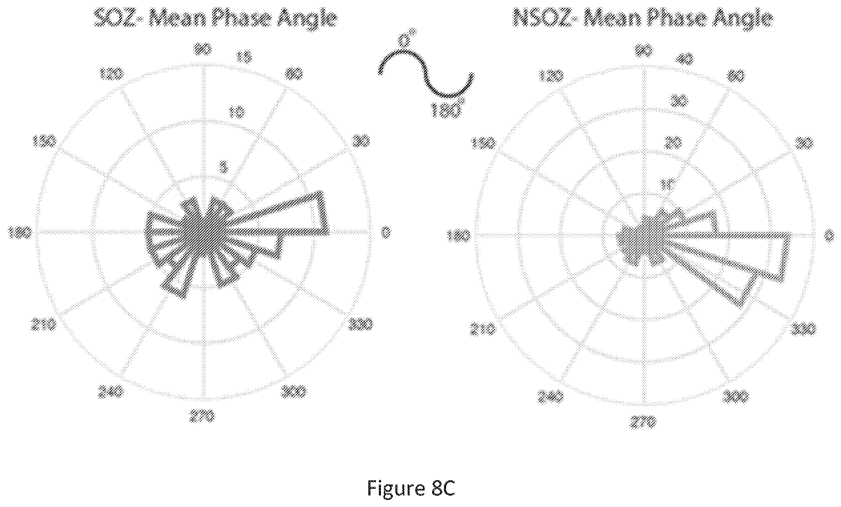

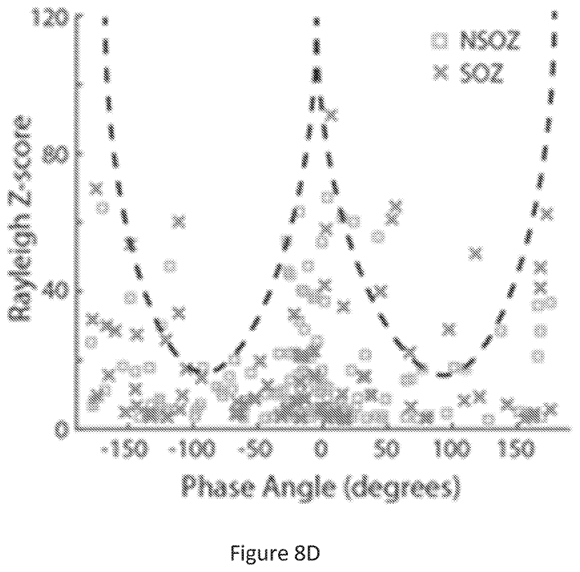

FIG. 8 illustrates that coupling between low frequency iEEG phase and ripple amplitude can be used to identify epileptogenic brain regions. In panel A, the ripple rate ratio is calculated on the basis of mean event rates measured from electrodes in the seizure onset zone (SOZ), and non-seizure onset zone (NSOZ). Both ripples and phase locked ripple phasors were precise biomarkers of the SOZ in all 11 patients. Across the 11 patients the ratio for phase locked ripple phasors exceeded the ratio for ripples (student's paired t-test, p<0.05). In panel B, correlation between biomarker expression and the probability a given macroelectrode lay within the SOZ. Within each patient, the biomarkers measured from each macroelectrode were rank ordered from greatest to least, SOZ probability was then determined for each rank ordered macroelectrode across patients on the basis of whether the assigned macroelectrode(s) was located in the SOZ. Ripple rate exhibited a strong correlation with epileptogenicity (B1), a correlation was also observed for the Rayleigh Z-statistic, a measure of phase locking strength between all the ripple phasors measured in a single macroelectrode recording (B2), phase locked ripple phasor rate also correlated with epileptogenicity, and macroelectrodes with the greatest rate, i.e. rank orders one and two, exhibited superior precision compared to ripples (B3). Panel C provides polar histogram of the mean phase angle of the phase locked ripples measured in the SOZ (C1) and the NSOZ (C2). A greater proportion of the ripples appeared to be coupled with the trough of the iEEG (4-30 Hz) in the SOZ relative to the NSOZ. Panel D provides a scatter plot of the mean phase angle and Rayleigh Z-score of ripple phasors identified in electrodes in the SOZ and in the (NSOZ). It is noted that phase locking strength was greatest at the peak or trough of the iEEG (dashed lines).

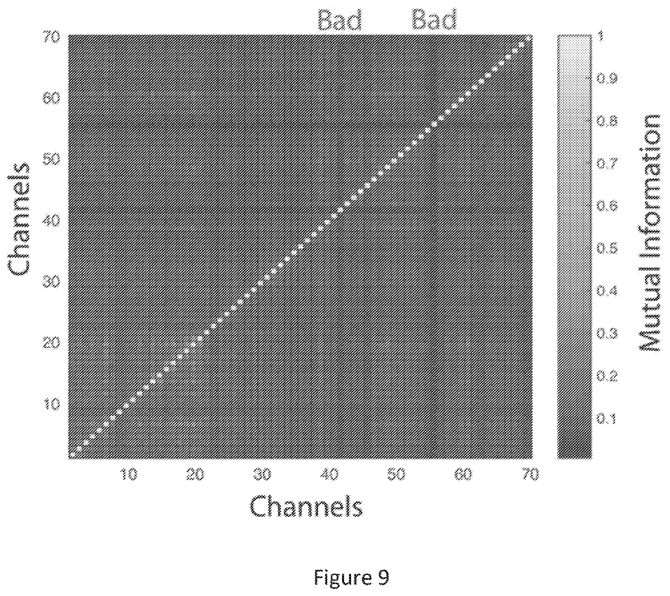

FIG. 9 depicts a mutual information adjacency matrix of the HFO (80-600) band pass filtered intracranial EEG calculated across all macroelectrode contacts during epochs of artifact. Recordings from electrode contacts exhibiting relatively low mutual information between multiple local and remote contacts are regarded as poor quality and excluded from further analysis to optimize ICA based artifact reduction and demarcation.

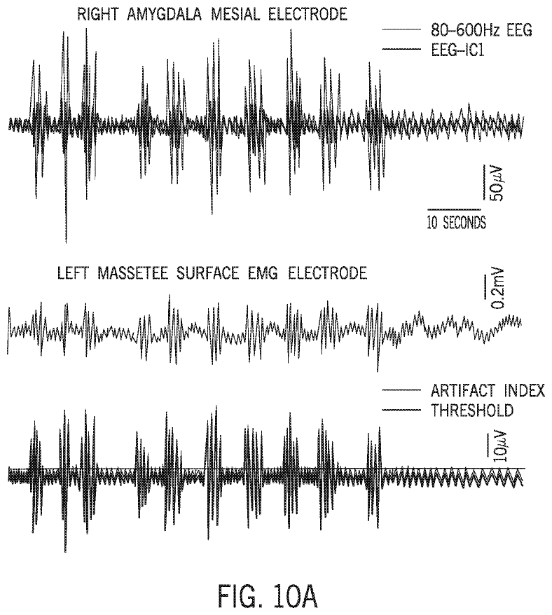

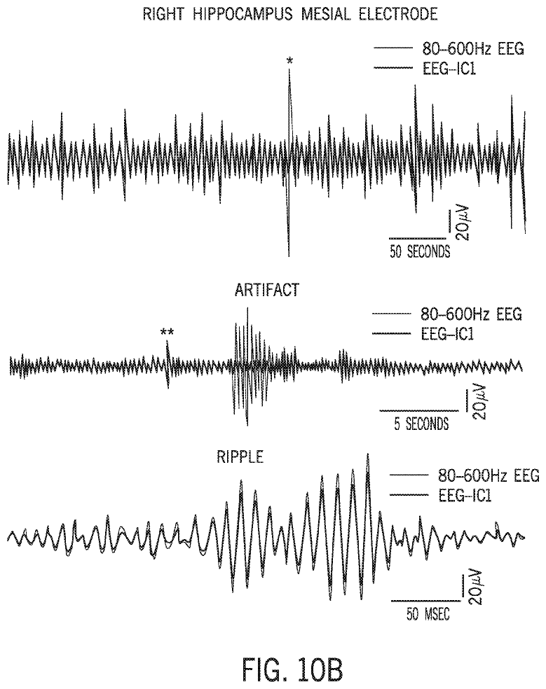

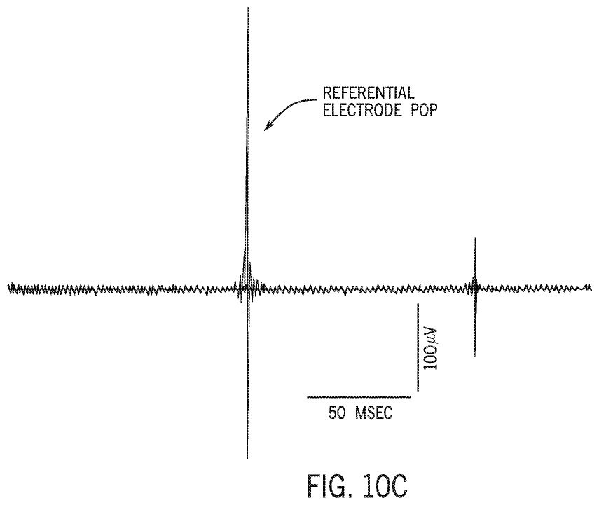

FIG. 10 illustrates that the first independent component (IC1) contains muscle and electrode artifact in the HFO band pass filtered intracranial EEG. Panel A depicts HFO band-pass filtered intracranial EEG recording from a mesial depth electrode contact during repetitive jaw clench (A1), and following pruning of IC1 (A2). Pruning IC1 reduced the muscle artifact in the iEEG recording but did not eliminate it. Artifact detection during this epoch containing residual artifact utilized the artifact index (A1) derived from IC1 (A3). As illustrated in panel B, pruning IC1 can remove muscle artifact from the intracranial EEG recording (B1, B2), while HFOs are unaffected (B3). Panel C shows electrode artifact can also be reduced by pruning IC1.

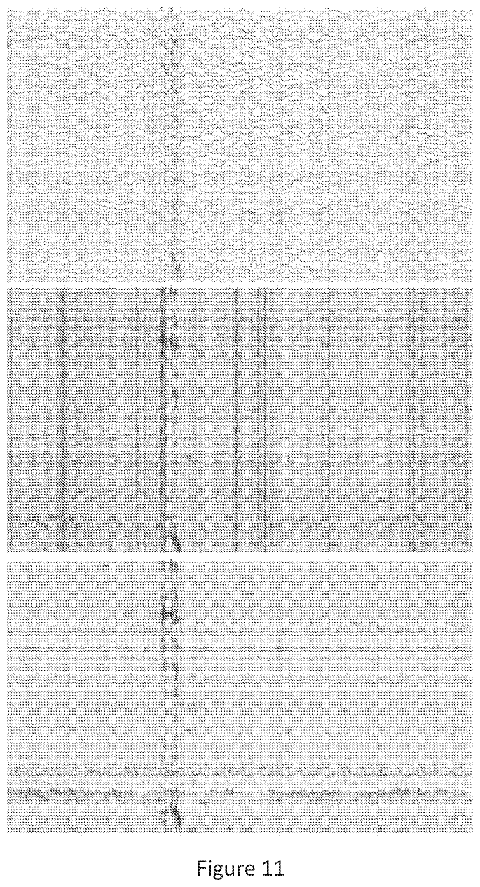

FIG. 11 illustrates that pruning the first independent component IC1 can markedly reduce muscle and electrode artifact across macroelectrode recording channels. Panel A depicts unfiltered intracranial EEG recordings from a subdural grid. Panel B shows the same recordings after applying a HFO (80-600 Hz) band pass filter demonstrating excessive muscle artifact between and during HFOs. Panel C shows the HFO band pass filtered EEG after pruning IC1 demonstrating clear HFOs and little residual artifact.

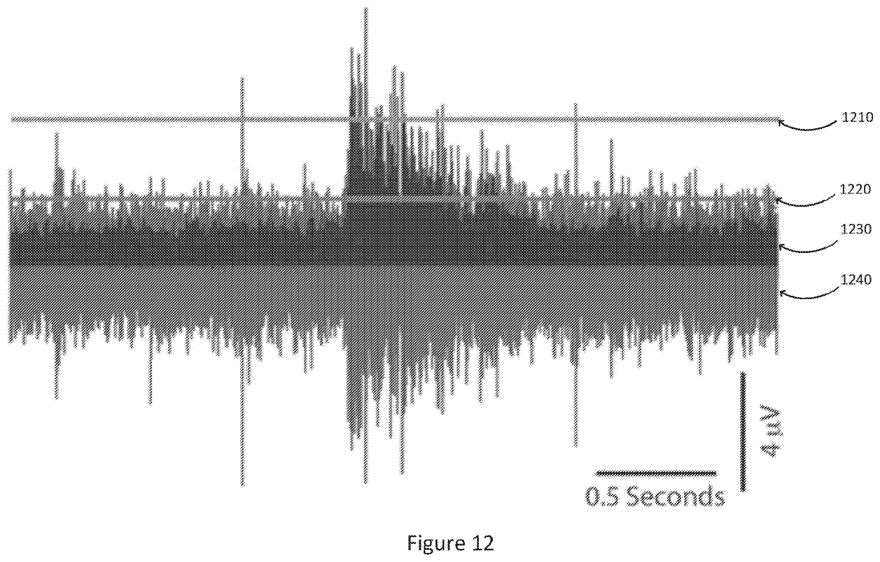

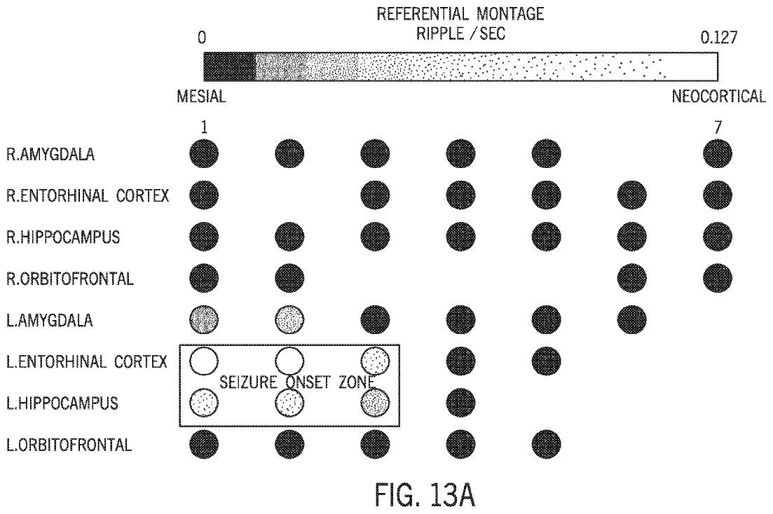

FIG. 12 provides an illustration of the utilization of the artifact index for the differentiation of brain HFOs from artifactual signal. A compressed time series of an HFO band pass filtered intracranial EEG recording from a mesial contact is depicted. When the artifact index crosses the threshold, increases in HFO amplitude above a second determined threshold are designated artifactual events.

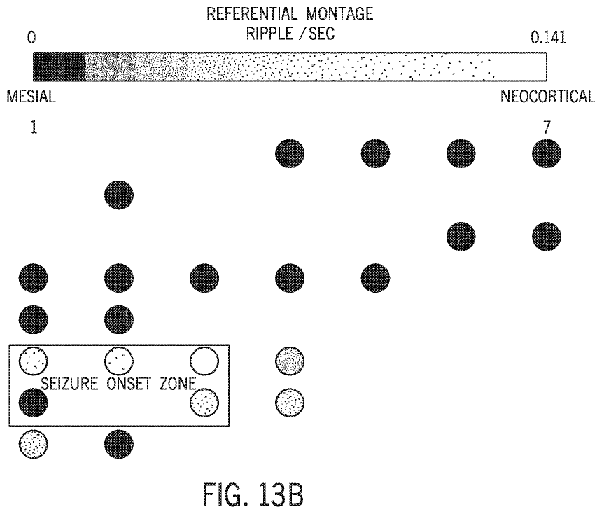

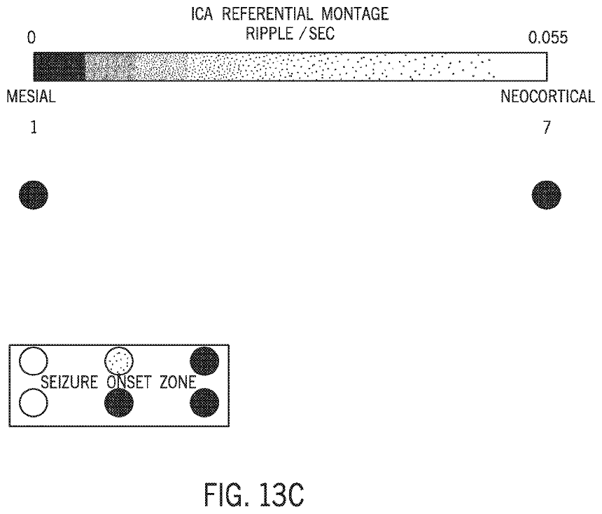

FIG. 13 illustrates that pruning independent component 1 and utilizing the artifact index during HFO detection for artifact differentiation improves the precision of event detection for delineating the seizure onset zone. Panel A provides a heat map of inter-ictal ripple rates across depth macroelectrode contacts in the referential montage. Panel B illustrates that HFO detection in a bipolar montage reduced HFO detection in contacts distant from the seizure onset zone. Panel C shows that HFO detection after both pruning IC1 and utilizing the artifact index demonstrated the greatest degree of precision for the seizure onset zone.

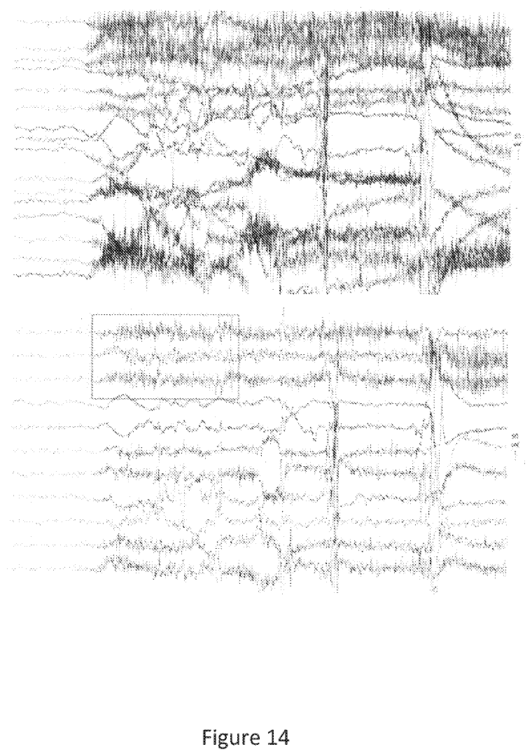

FIG. 14 illustrates that exemplary implementations improve the clarity of visual inspection and interpretation of traditional scalp EEG. Panel A provides a recording of a seizure from a patient with suspected nocturnal frontal lobe epilepsy with a semiology consistent with a left hemisphere onset. The record is nearly completely obscured by muscle artifact. Panel B shows the same EEG after application of an exemplary process, illustrating the clear ictal rhythm at onset in the left frontal lobe.

To further illustrate the exemplary implementations, a study aimed at determining if coupling between low frequency (4-30 Hz) iEEG phase and ripple (80-150 Hz) amplitude is exaggerated in mesial temporal epileptogenic regions will now be discussed. Ripples in inter-ictal intracranial depth macroelectrode recordings from 11 patients with mesial temporal lobe epilepsy (MTLE) were analyzed using an automated and unsupervised detector with built in artifact reduction and exclusion capabilities. Phase amplitude coupling (PAC) was investigated by transforming each ripple in to a corresponding phasor on the basis of its relationship with the low frequency iEEG. The results showed that computer aided visual inspection of the iEEG revealed two forms of PAC, type I: coupling between inter-ictal discharge phase and ripple amplitude, and type II: coupling between theta oscillation phase and ripple amplitude. These two types of PAC could be expressed exclusively or as a mixture in hippocampus, entorhinal cortex, amygdala, or neocortex. Ripple rates calculated irrespective of phase exhibited a 78.2% precision for the seizure onset zone (SOZ), and were increased in the SOZ relative to the non-SOZ in all 11 patients. Phase locking strength of the corresponding ripple phasors was also increased in the seizure onset zone, and the phase locked ripple phasor rate ratios for the SOZ were increased relative to the ripple rate ratios (student's paired t-test, p<0.05). It is shown that ripple rates in intracranial depth macroelectrode recordings measured with an innovative detector with built in artifact rejection are a precise biomarker of mesial temporal epileptogenic regions. Coupling between the phase of the low frequency iEEG and ripple amplitude is exaggerated in these regions and can be used to more precisely delineate the seizure onset zone.

MTLE is the most common form of medically refractory epilepsy, and is often amenable to respective epilepsy surgery. When the seizure onset zone is unilateral anterior temporal lobectomy is the recommended procedure and up to 80% of patients have seizure free post-operative outcomes. Recently, implantation of a repetitive nerve stimulation (RNS) device has become the standard of care for MTLE patients with bilateral independent seizure onset zones. Differentiating MTLE patients with primarily unilateral seizure onsets from patients with bilateral independent seizure onsets would be aided by a precise neurophysiological biomarker of epileptogenic mesial temporal lobe brain tissue. High frequency oscillations (HFOs) are brief (15-200 milliseconds) bursts of neurophysiological activity that range in frequency between 80 and 600 Hz, and are thought to serve as a precise biomarker of epileptogenic brain. Epileptogenic hippocampus and entorhinal cortex can be identified on the basis of increased fast ripple (200-600 Hz) HFO rates, but not increased ripple (80-200 Hz) HFO rates in microelectrode recordings. However, in recordings from clinical macroelectrodes, either increased rates of ripples or fast ripples identify the epileptogenic hippocampus and entorhinal cortex.

Ripples are generated during memory formation and consolidation in the hippocampus. The characteristics that distinguish physiological ripples from pathophysiological ripples remain unknown. In this study we asked if coupling between the phase of the low frequency (4-30 Hz) iEEG and the amplitude of ripple (80-150 Hz) oscillations would serve as a precise biomarker of epileptogenic mesial temporal lobe brain tissue by distinguishing pathological ripple events. To approach this problem we utilized a recently developed automated and unsupervised HFO detector with advanced artifact reduction and exclusion capabilities.

Consecutive patients who underwent intracranial evaluation at the UCLA seizure disorder center with depth electrodes between 2010-2015 were selected on the basis of 1) the availability of inter-ictal recordings obtained with a 2 kHz sampling rate of at least a 5 minute duration, and 2) a seizure onset zone in the mesial temporal lobe grey matter confirmed by macroelectrodes implanted there. Clinical depth intracranial EEG (iEEG, 0.1-600 Hz; 2000 samples/second; reference scalp Fz) was recorded using a Stellate (XLTEK, San Diego, Calif., U.S.A) or a Nihon-Kohden 128-channel NK 1200 long-term monitoring system (Nihon-Kohden America, Foothill Ranch, Calif., U.S.A.). For the recordings obtained using the Stellate system only one data segment was available for each patient, in some patients the data segment was acquired during wakefulness, but in other patients the data segment was obtained during drowsiness and/or sleep. For the recordings obtained using the Nihon-Kohden system continuous recordings were available and data segments one hour in duration were selected during the early morning (02:00-04:00) during drowsiness and/or sleep. Visual inspection of the data revealed that muscle artifact was evident in all the recording segments to varying degrees.

Ripple detections in the iEEG recordings were carried out using custom software developed in Matlab (Mathworks, Natick, Mass.). The recordings obtained on the Stellate and Nihon-Kohden systems were exported as European Data Format (EDF) and imported in to Matlab. During some epochs the iEEG recordings were completely obscured by high amplitude muscle artifact for extended durations often corresponding to physical activity. To exclude these segments from further analysis the power spectral density (PSD) in the ripple (80-150 Hz) band was computed in 90 second bins from a manually selected macroelectrode recording. Epochs containing excessive and prolonged muscle artifact were identified on the basis of aberrantly large ripple power and excluded from further analysis on the basis of applying a threshold to the measured PSD values.

This method could not exclude or reduce brief epochs of muscle or electrode artifact during otherwise high quality recording epochs. A unique algorithm was developed based on independent component analysis (ICA) to reduce high frequency artifact in the iEEG, and differentiate the increases in high frequency amplitude generated by artifactual sources from brain generated HFOs with a sub-millisecond time resolution. This exemplary method also served to identify intracranial electrode contacts with poor recording quality from further analysis.

After applying this ICA based method, ripples were detected in the iEEG macroelectrode recordings by: 1) applying a 1000th order symmetric finite impulse response (FIR) band pass (80-600 Hz) filter to the iEEG recording from a single macroelectrode contact; 2) applying a Hilbert transform to calculate the instantaneous amplitude of this time series according to the analytic signal z(t) described in the equation:

z(t)=a(t)e^{iϕ(t)} (2);

where a(t) is the instantaneous amplitude of z(t), and phi(t) is the instantaneous phase of z(t); 3) smoothing the instantaneous HFO amplitude function using moving window averaging; 4) normalizing the smoothed instantaneous HFO amplitude function based on the mean and standard deviation of the time series; and 5) applying a threshold to detect the onset and offset of discrete events.

For each detected event, a power spectral density function was used to calculate the mean frequency. A minimum duration criteria was applied to each detected event with a threshold based on its mean frequency, events that did not meet the criteria were excluded on the basis that the brief increases in high frequency amplitude did not correspond to a HFO event with at least 3-4 cycles of the signal. Events occurring during epochs containing artifact, defined on the basis of the PSD method that identified entire data blocks or the ICA method that identified epochs as short as a millisecond, were also excluded.

HFOs were iteratively detected across all the intracranial macroelectrodes in twenty second bins, and only ripple events with a frequency between (80-150 Hz) were subject to further statistical analysis in this study.

To examine phase amplitude coupling between HFO events and the low frequency (4-30 Hz) iEEG recording, we applied an automated method based on transforming each distinct HFO event in to a HFO phasor calculated on the basis of the instantaneous phase of the low frequency iEEG recording and the corresponding instantaneous amplitude during the duration of the HFO event.

The instantaneous phase of the low frequency iEEG during HFO events was calculated by: 1) applying a 1000th order FIR bandpass (4-30 Hz) filter to the iEEG recording; 2) applying a Hilbert transform to this time series and calculating phi(t); and 3) saving phi(t) values that correspond to HFO epochs for further analysis.

Each HFO phasor was calculated using the equation:

$${ve}^{i θ} = {\sum }_{t}^{T} a ( t ) {e}^{i φ ( t )} ; \qquad ( 3 )$$

where v is the vector strength of the phasor, theta its phase angle, and a(t) and phi(t) are the respective instantaneous HFO amplitude and low frequency iEEG phase during the HFO across its duration. Phase locking among the multiple HFO phasors [n . . . N] detected from individual macroelectrodes was performed using Rayleigh's test for circular non-uniformity by calculating the mean vector strength (r) using:

$$r = \frac{\vert {\sum }_{n}^{N} {v}_{n} {e}^{i {θ}_{n}} \vert}{{\sum }_{n}^{N} {v}_{n}} ; \qquad ( 4 )$$

and deriving the Rayleigh Z-statistic using:

Z=Nr² (5).

Phase amplitude coupling was also characterized by calculating modulation index (M) values using a surrogate measure approach. Raw modulation index is defined by:

$$M = \vert {\sum }_{t}^{T} a ( t ) {e}^{i φ ( t )} \vert ; \qquad ( 6 )$$

where a(t) is the instantaneous amplitude during the duration of the recording[t . . . T], and phi(t) is the instantaneous phase. To calculate modulation index across varying frequencies, phi(t) was calculated using a 1000th order FIR bandpass filter in 0.5 Hz bins between 2 and 22 Hz, and a(t) was calculated using the same filter in 5 Hz bins between 20-240 Hz.

To investigate phase amplitude coupling of ripple oscillations detected from intracranial macroelectrodes in mesial temporal lobe epilepsy, we applied an automatic and unsupervised ripple detector to 5-60 minute intracranial recordings from 591 macroelectrode contacts during wakefulness and sleep. The detector was equipped to reduce and exclude epochs of increased high frequency activity generated by muscle and electrode artifact (further discussed below). Overall, a total of 27,826 ripples were detected.

In a subset of the macroelectrode recordings, the detector results were used to annotate the iEEG recording. The overall sensitivity of the detector for HFO events during epochs containing artifact was 96.57%+/−2%, and the overall precision of the detector was 88.74+/−6.5% (n=4) as compared to the gold standard of visual inspection. Furthermore, the ratio of the ripple rates detected from macroelectrodes implanted in gray matter relative to the ripple rates detected from macroelectrodes in white matter was 4.03+/−1.85 (n=7, paired student's t-test p<0.05).

The results of the automatic detector were used for subsequent visual inspection of both the detected ripples (80-150 Hz), and the corresponding low frequency iEEG (4-30 Hz) activity. Two types of coupling between the low frequency iEEG phase and ripple amplitude were identified in this analysis. In the first type (Type I), the phase of inter-ictal epileptiform discharges were coupled with the amplitude of the corresponding ripple events (see FIG. 5, panels A, B). To assess if this phase amplitude relationship remained consistent across all the ripples in the recording, each individual ripple was transformed in to a phasor on the basis of its phase-amplitude relationship with the low frequency iEEG. Subsequently, the ripple phasors were compared and it was assessed if they statistically clustered around a mean phase angle, i.e., phase lock (see FIG. 5, panel C)

A second type of coupling (Type II) was evident between the phase of theta oscillations in the low frequency iEEG and ripple amplitude (FIG. 5, panels A, B). The ripple events in this type of phase-amplitude coupling were often of a longer duration as compared to ripples associated with inter-ictal discharges. In this case, the corresponding ripple phasors also exhibited phase locking around a common phase angle (FIG. 5, panel C).

Both types of phase amplitude coupling were sometimes evident in the hippocampus, amygdala, entorhinal cortex, parahippocampal gyrus, and also the neocortex. The two types of phase amplitude coupling were not mutually exclusive, in recordings from some intracranial electrodes both types were intermixed but the ripple phasors corresponding to both types shared a common mean phase angle. We did not specifically examine coupling between iEEG phase <4 Hz and ripple amplitude.

Visual inspection of the data also suggested that coupling between low frequency iEEG phase and ripple amplitude was more often seen in the seizure onset zone. Some electrodes outside the seizure onset zone exhibited ripples but the corresponding phasors did not cluster around a mean phase angle (see FIG. 5).

To determine if phase amplitude coupling was consistent across the entire recording duration, and specifically examine the frequencies involved, we calculated the modulation index between low frequency iEEG phase in 0.5 Hz bins, and higher frequencies iEEG amplitude in 5 Hz bins in one hour inter-ictal recordings. In macroelectrode recordings that exhibited coupling between inter-ictal discharge phase and ripple amplitude on visual inspection, the modulation index reflected strong phase-amplitude coupling between theta band phase (4-7 Hz), and gamma (30-80 Hz) as well as ripple (90-110 Hz) amplitude. Theta band phase (4-7 Hz) was also coupled with higher frequency (120-130 Hz) and (180-190 Hz) ripple amplitude. The modulation index was also increased between alpha band phase (9-13 Hz) and gamma and ripple amplitude (FIG. 6, left panel).

The presence of “islands” in the calculated modulation index values reflect increased modulation between theta and alpha band phase and specific sub-bands of gamma and ripple amplitude (FIG. 6, left panel). If coupling between epileptiform discharge phase and ripple oscillation amplitude was due to a spurious correlation resulting from Gibbs phenomena, the modulation index would not exhibit “islands” but rather a continuous gradient across the gamma and ripple band.

In macroelectrode recordings that exhibited coupling between theta phase and ripple amplitude on visual inspection, the modulation index reflected strong phase-amplitude coupling between the theta band phase (4-7 Hz), and gamma (35-55 Hz and 65-75 Hz) as well as ripple (90-100 Hz) amplitude. No coupling was evident between theta band phase and ripple amplitude at frequencies >100 Hz (FIG. 6, middle panel). When the modulation index was calculated using recordings from macroelectrodes located outside the seizure onset zone that exhibited ripples, but no clear phase amplitude coupling, no increases in modulation index were evident (FIG. 6, right panel).

We tested whether ripples that were coupled with the phase of the low frequency iEEG would provide any advantage in localizing epileptogenic regions in comparison to ripples detected irrespective of the phase of the low frequency iEEG (see FIG. 7, panels A1, A2, B). To perform this comparison we calculated the phase locked ripple phasor rate on the basis of 1) identifying electrodes in which the ripple phasors for all the detected ripple events exhibited statistically significant phase locking (Rayleigh's test for circular non-uniformity, p<0.05), and 2) tallying the number of ripple phasors that were within +/−90 degrees of the mean phase angle of the total population of ripple phasors (see FIG. 7, panels C1, C2).

In each of the 11 patients we found that the mean ripple rates in recordings from macroelectrodes in the seizure onset zone (SOZ) exceeded the mean ripple rates calculated from macroelectrodes outside the seizure onset zone (NSOZ) (see FIG. 8, panel A). The overall precision of ripple rates for the seizure onset zone was 78.2% at a normalized threshold of 2.5 standard deviations above the mean. When the rate ratio was calculated on the basis of phase locked ripple phasor rates measured from macroelectrodes in the SOZ relative to the NSOZ, a relative increase in the ratio was seen across the eleven patients relative to the ripple rate (student's t-test p<0.05) (see FIG. 8, panel A).

Examining the difference in mean biomarker, i.e., ripple event rates, in the SOZ and NSOZ rates is problematic because the strength of each biomarker at every individual electrode site is not explicitly compared. To correlate biomarker strength with epileptogenicity at every electrode site, the strength of the biomarker (i.e., rate) measured for each macroelectrode contact from a single patient was rank ordered from greatest to least. The probability that these rank ordered electrodes were located in the seizure onset zone was determined across patients on the basis of whether or not the respective electrode was located in the seizure onset zone in each patient.

A clear correlation was seen between rank ordered ripple rate and the probability the respective electrodes were located in the SOZ (see FIG. 8, panel B1). In 10 of the 11 patients the macroelectrode exhibiting the greatest ripple rate was implanted in the seizure onset zone. This was true for patient #8 in whom inter-ictal discharge frequency was greatest in macroelectrodes contralateral to the seizure onset zone. This was also the case for patient 431 with a primarily lateral temporal seizure onset zone and corresponding greatest ripple rate at this site. In patient #4, whom exhibited bilateral independent mesial-temporal lobe seizure onsets but underwent a right anterior temporal lobectomy with an Engel II outcome the macroelectrodes with the greatest ripple rates was in the right amygdala and hippocampus. In patient #9 the macroelectrode exhibiting the greatest ripple rate was located in the inferior frontal gyrus outside of the seizure onset zone. Visual inspection of the annotated ripple events in the recording from this macroelectrode demonstrated that the ripples were relatively prolonged relative to ripples detected in mesial temporal structures, and the events may have been of a physiological rather than pathological nature.

To determine if the strength of phase amplitude coupling correlates with epileptogenicity, we compared the rank ordered Rayleigh's Z-statistic, a measure of phase locking strength, to seizure onset zone probability. The Rayleigh Z-statistic is not a direct measure of the number of ripple events, but rather how tightly the corresponding ripple phasors cluster around a common phase angle. We found that macroelectrodes with a larger Z-statistic were more likely to be located in the seizure onset zone (see FIG. 8, panel B2). However, in contrast to overall ripple rate, for 3 of the 11 patients the macroelectrode with the greatest Rayleigh Z-statistic were located outside of the seizure onset zone.

The phase locked ripple phasor rates are calculated on the basis of both overall ripple rate and the strength of phase locking among the ripple phasors. We found that for all 11 patients the macroelectrode with the greatest phase locked ripple phasor rate resided within the seizure onset zone (see FIG. 8, panel B3).

We also examined if the properties of coupling between the phase of the low frequency iEEG and ripple amplitude differed between electrodes located in the SOZ and NSOZ. Polar histograms of the mean phase angle of the phase locked ripple phasors measured across all the macroelectrodes located in the SOZ revealed a bimodal distribution with most of the electrodes exhibiting ripples phase locked near either the trough or the peak of the low frequency iEEG phase (see FIG. 8, panel C1). In contrast, the corresponding polar histogram for the macroelectrodes located in the NSOZ revealed a largely unimodal distribution with most of the electrodes exhibiting ripple phase locked to the peak of the low frequency iEEG phase (see FIG. 8, panel C2).

Comparing the Rayleigh Z-statistic with the mean phase angle for each macroelectrode in the SOZ and NSOZ revealed that phase locking strength was greatest when the mean phase angle was near either the peak or the trough of the low frequency iEEG. This relationship was independent of whether or not the macroelectrode resided in the SOZ or NSOZ (see FIG. 8, panel D).

This study identified two distinct types of coupling between the phase of the low frequency iEEG and the amplitude of ripple oscillations: Type I in which inter-ictal discharge phase was coupled with ripple amplitude, and Type II in which theta oscillation phase was coupled with ripple amplitude. Both types were evident in recordings from the seizure onset zone. Also, the study provided confirmation that the strength of coupling, and the related phase locked ripple phasor rate, are both elevated in epileptogenic mesial temporal lobe grey matter.

We asked if coupling between the phase of the low frequency iEEG and ripple amplitude could be used to distinguish ripple events of pathological significance from physiological ripples. We found that ripple rates calculated irrespective of phase were precise for the seizure onset zone. The precision of the ripple rates in this study provides a benchmark to compare our HFO detector to others that offer artifact rejection. Further, this finding suggests that ripples recorded in macroelectrode iEEG recordings from MTLE patients are of pathophysiological significance.

In most patients we observed exaggerated coupling between the phase of the low frequency iEEG and ripple amplitude in the seizure onset zone. Also, the phase locked ripple phasor rate outperformed the ripple rate in distinguishing epileptogenic brain regions in several patients. Together these findings suggest that when ripples are detected in depth macroelectrode recordings they are probably generated by a pathophysiological mechanism, but this mechanism may involve increased phase amplitude coupling.

Physiological ripples, associated with memory formation and consolidation, have been characterized in microelectrode recordings from the hippocampus of rodents and humans. Although it has been demonstrated that these ripples are generated across large regions of the hippocampus, our macroelectrode recordings suggest that these events are either too infrequent, or too small in amplitude, to introduce significant false positive results that interfere with delineation of the seizure onset zone. Future detailed microelectrode studies could test this hypothesis by examining if ripples in epileptogenic mesial temporal grey matter are detected across larger regions relative to ripples in healthy tissue. An increase in the size and strength of the ripple generator in epileptogenic mesial temporal grey matter, relative to healthy tissue, could explain why ripple rates did not localize epileptogenic regions in microelectrode recordings from the human hippocampus, but were localizing in macroelectrode recordings.

Phase amplitude coupling is not explicitly a pathophysiological phenomenon. In fact coupling between the amplitude of high gamma (80-150 Hz) oscillatory activity and the phase of the EEG occurs during normal cognition in the cortex. In certain cases, such as during a seizure, coupling between the low frequency ictal rhythm and ripple activity has been proposed to represent a transient translation of increased synaptic drive into spiking output.

Amplification of phase amplitude coupling could reflect multiple pathological processes including disrupted excitatory/inhibitory balance, a disorganization or reorganization of neural circuits, or abnormal synchronization of networks. One possibility is that in epileptogenic brain regions the increased excitatory synaptic drive during either inter-ictal discharges or theta oscillatory activity produces synchronized bursts of action potentials across large volumes of tissue resulting in a ripple oscillation that can be detected in a macroelectrode. This would explain why most ripple phasors were phase locked to either the peak or the trough of the low frequency EEG when the synaptic drive is maximal. While ripples recorded from electrodes in the SOZ were more likely to be phase locked to the trough, rather than the peak, of the low frequency iEEG, the difference was likely due to the relative spatial geometry of the dipole of the synaptic generator. Future investigations should also utilize microelectrode recordings to characterize how spiking among excitatory and inhibitory single units relates to coupling of LFP phase with ripple amplitude.

Ripple rates were thus found to be a precise biomarker for the seizure onset zone for MTLE patients, and coupling between the low frequency iEEG phase and ripple amplitude is often exaggerated in epileptogenic regions. An improved understanding of the role of pathophysiological phase amplitude coupling in epilepsy may be essential for defining the network changes that accompany epileptogenesis. Furthermore, clinically utilizing both ripple rates and phase amplitude coupling strength could potentially improve surgical seizure outcomes by identifying patients with bilateral MTLE, who would derive greater benefit from an RNS implant as compared with anterior temporal lobectomy.

To reduce artifact for HFO detection, an ICA-based artifact reduction method for intracranial EEG is based on two assumptions: 1) that more variance in the HFO (80-600 Hz) band pass filtered intracranial EEG across electrodes is accounted for by muscle and electrode artifact than brain potentials; and 2) that the performance and reliability of ICA to separate muscle and electrode artifact from brain potentials will improve if the mutual information between all the HFO band pass filtered intracranial EEG signals is sufficiently large during artifactual epochs (see FIG. 9).

Using ICA algorithms in which a decreasing order of the HFO band pass filtered EEG variance is accounted for by each additional independent component, such as Infomax ICA, will result in the first independent components accounting for muscle and electrode artifact, while the higher components account for brain potentials.

For the removal of muscle and electrode artifact from inter-ictal HFO band pass filtered intracranial recordings, for the purposes of HFO detection and characterization, the first independent component typically accounts for the vast majority of artifactual signal (see FIG. 10). Pruning the first independent component from the HFO band pass filtered EEG recording reduces and sometime eliminates muscle and electrode artifact, but does not influence the integrity of inter-ictal brain generated HFO waveforms. However, in some cases a residual artifactual signal may remain (see FIGS. 10, 11).

To detect high frequency activity that is of an artifactual nature after pruning the first independent component, an artifact index is derived using Eqn. 7 below, where AI(t) is the artifact index, hfo(t) is the band pass filtered EEG or LFP recording, and ic1(t) is the band pass filtered EEG or LFP recording after pruning the first independent component (see FIGS. 9, 10 (A3)), and T is the total number of points in the time series.

$$AI ( t ) = \frac{\vert hfo ( t ) - ic 1 ( t ) \vert}{( Σ \vert hfo ( t ) \vert ) \text{/} T} or AI ( t ) = \frac{\vert {hfo}_{i} ( t ) - ic {1}_{i} ( t ) \vert}{Σ \vert {hfo}_{i} ( t ) \vert \text{/} T} * \frac{1}{I} {Σ}_{i}^{I} \frac{\vert {hfo}_{i} ( t ) - ic {1}_{i} ( t ) \vert}{Σ \vert {hfo}_{i} ( t ) \vert \text{/} T} . \qquad ( 7 )$$

During the detection of discrete increases in high frequency amplitude in the ic1(t) time series, artifactual increases in high frequency amplitude can be distinguished from brain generated HFOs on the basis of AI(t) exceeding a threshold, i.e. 3.5, at any time point during the duration of the HFO event (FIGS. 12, 13).

In the study, computations were carried out using compiled Matlab 8.4 custom scripts on a cluster of HP SL230s Gen 8 ES-2670 nodes with dual-eight-core 2.6 GHz Intel ES-2670 central processing units, 4 GB of memory per core, and NVIDIA Tesla graphics processing units. Inter-ictal intracranial data recordings sampled at 2 kHz and up to one hour in duration were imported as EDF files. All channels of the remaining intracranial EEG were band pass filtered (80-150 Hz), and a simple algorithm designed to find extended intervals of elevated high frequency amplitude across electrodes was used to select for more subtle artifacts in the recording that were undetected using power spectral density analysis.

The normalized mutual information adjacency matrix was calculated across all macroelectrode contacts during the (80-150 Hz) band pass filtered artifact epoch of greatest duration. Each macroelectrode was assigned a single mutual information value by calculating the mode of the largest 21 respective mutual information pair values. If this mutual information value did not exceed a threshold, its respective electrode was deemed a poor recording contact and was excluded from further analysis. The band pass filtered (80-600 Hz) intracranial EEG was then binned into 90-second trials, and each trial was processed using CUDAICA, an Infomax ICA algorithm that operates on a paired CPU and GPU and significantly reduces processing time. The first independent component of the band pass (80-600 Hz) filtered EEG was pruned, and the artifact index was calculated to optimize the differentiation of artifact from HFOs generated by brain. Automated and unsupervised HFO detection using the Hilbert based detector, as described above, was performed on the pruned band pass (80-600 Hz) filtered EEG i.e. ic1(t) utilizing the AI(t) time series. The complete analysis of 64 channels with a 2 kHz sample rate of one hour duration was typically completed within two to three hours.

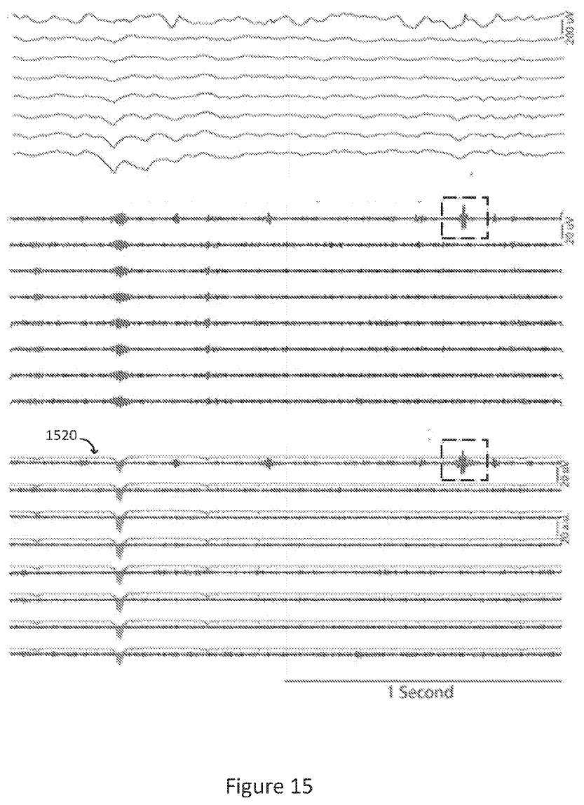

Referring to FIG. 15, an iEEG recording from eight contacts in referential montage (top) demonstrates no obvious artifact. Band-pass (80-200 Hz) filtering (middle) reveals a ripple occurring synchronously in all the recordings, followed by a ripple with an unconvincing morphology in LASP4 (dashed box). Performing ICA on the band-pass filtered signal and pruning independent component 1 (bottom) results in the elimination of the first ripple in all the recordings, and the unmasking of additional oscillatory cycles in the subsequent ripple in LASP4 (dashed box). The artifact index (1520) indicates the epoch in which the artefactual HFO contaminated the recording.

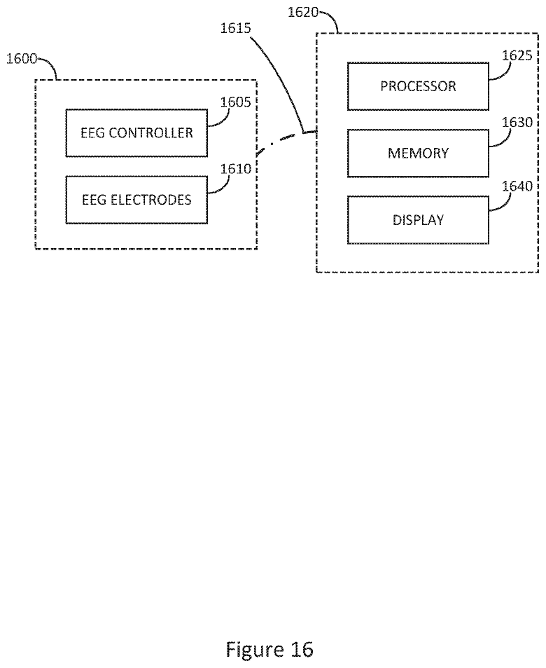

Referring to FIG. 16, exemplary systems implementing the above approaches may include an EEG device 1600 with an EEG controller 1605 and EEG electrodes 1610. The EEG controller 1605 may direct the activation of the EEG electrodes 1610 and acquisition of electrical signals therefrom. The EEG device may itself be capable of processing, interpreting, and/or analyzing the electrical signals received, or alternatively, it may be connected via communications interface 1615 to a computing device 1620. The communications interface 1615 may be any wired or wireless method or protocol for the exchange of information between EEG device 1600 and the computing device 1620. If the computing device 1620 is a remote server, for example, the communications interface 1615 may involve a wired or wireless connection capable of communicating information to the remote server via one or more networking protocols, such as those used by the Internet. The computing device 2020 may receive information on the electrical signals acquired using the EEG electrodes 2010 and transmitted by the EEG device 1600.

The computing device 1620 may include a processor 1625 and a memory 1630 as a digital storage capability with instructions (software, code, etc.) that can be executed by the processor. For example, the memory 1630 may include code that directs the computing device to process the electrical signals received from the EEG device 1600 to implement one or more of the above exemplary processes for artifact reduction. The computing device 1620 may also include a display 1640 for presentation of data or results. For example, the display may be used for presenting montages for visual inspection and interpretation by healthcare providers and researchers. The display 1640 may also present indices and other quantifications corresponding with, or representative of, the electrical signals acquired via the EEG electrodes 1610 and analyzed by computing device 1620.

Implementations of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Further, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure(s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, computer processor, central processing unit (CPU), and computer are used synonymously to denote a device capable of executing the instructions and communicating with input/output interfaces and/or peripheral devices, and that the terms processor, computer processor, CPU, and computer are intended to encompass single or multiple devices, single core and multicore devices, and variations thereof.

Although the description herein contains many details, these should not be construed as limiting the scope of the disclosure but as merely providing illustrations of some of the presently preferred implementations. Therefore, it will be appreciated that the scope of the disclosure fully encompasses other configurations which may become obvious to those skilled in the art.

In the claims, reference to an element in the singular is not intended to mean “one and only one” unless explicitly so stated, but rather “one or more.” All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

The present invention has been described in terms of one or more preferred versions, and it should be appreciated that many equivalents, alternatives, variations, additions, and modifications, aside from those expressly stated, and apart from combining the different features of the foregoing versions in varying ways, can be made and are within the scope of the invention. The above systems and methods can be implemented using hardware, software, single integrated devices, multiple devices in wired or wireless communication, or any combination thereof.

1. A method for reducing artifacts in neural activity readings, the method including:
receiving electrical readings acquired using multiple EEG electrodes secured to a subject;
applying, in consecutive iterations, independent component analysis (ICA) to the electrical readings;
pruning selected components in each iteration to produce reduced-artifact electrical readings; and
reporting on the reduced-artifact electrical readings.

2. The method of claim 1, wherein pruning selected components includes:
calculating an inverse weight matrix; and
pruning one or more independent components with inverse weight matrix values which exceed a matrix threshold.

3. The method of claim 1, wherein the electrical readings are filtered to favor frequencies at or above 16 Hz.

4. The method of claim 1, further including:
applying a low-pass filter to the received electrical readings to obtain low-pass filtered readings; and
reconstituting the low-pass filtered readings with the filtered and pruned electrical readings.

5. The method of claim 4, wherein the EEG electrodes are scalp electrodes.

6. The method of claim 1, wherein pruning selected components includes pruning a first independent component (IC1) in each consecutive iteration.

7. The method of claim 6, wherein the EEG electrodes are intracranial.

8. The method of claim 1, further including concatenating one or more EEG recordings after pruning independent components.

9. (canceled)

10. The method of claim 1, further including determining which EEG electrodes recorded brain activity with relatively low fidelity and excluding data thereof.

11. The method of claim 10, wherein determining which EEG electrodes recorded brain activity with relatively low fidelity and excluding data thereof includes:
filtering the electrical readings using a symmetric digital finite impulse response (FIR) filter;
identifying an artifact epoch based on normalized mean amplitude across electrodes;
calculating mutual information (MI) across electrodes in the artifact epoch; and identifying electrodes with MI below an MI threshold and excluding corresponding channels from the electrical readings.

12. The method of claim 10, wherein determining which EEG electrodes recorded brain activity with relatively low fidelity and excluding data thereof includes:
calculating a mutual information association matrix across electrodes during an epoch, the mutual information association matrix having mutual information values for the recording electrodes;
identifying as faulty any electrodes with mutual information values below a mutual information threshold; and
excluding data acquired using faulty electrodes from the electrical readings to be analyzed and pruned.

13. A system for reducing artifacts in neural activity readings, the system including a processor and memory with instructions thereon, the processor being configured to:
receive electrical readings acquired using multiple EEG electrodes secured to a subject;
apply a high-pass filter to favor electrical readings with frequencies above a high threshold;
apply, in consecutive iterations, independent component analysis (ICA) to the high-pass filtered electrical readings;
prune selected components in each iteration to generate reduced-artifact electrical readings; and
produce a report of the reduced-artifact electrical readings.

14. The system of claim 13, wherein pruning selected components includes the processor being configured to:
calculate an inverse weight matrix; and
prune one or more independent components with inverse weight matrix values which exceed a matrix threshold.

15. The system of claim 13, wherein the EEG electrodes are intracranial, and wherein pruning selected components includes the processor being configured to prune a first independent component (IC1) in each consecutive iteration.

16. The system of claim 13, wherein the EEG electrodes are scalp electrodes, and wherein the processor is further configured to:
apply a low-pass filter to the received electrical readings to obtain low-pass filtered readings; and
reconstitute the low-pass filtered readings with the high-pass filtered and pruned electrical readings.

17. The system of claim 13, wherein the processor is further configured to concatenate one or more EEG records after pruning independent components.

18. (canceled)

19. The system of claim 13, wherein the processor is further configured to:
filter the electrical readings using a symmetric digital finite impulse response (FIR) filter;
identify an artifact epoch based on normalized mean amplitude across electrodes; calculate mutual information (MI) across electrodes in the artifact epoch; and
identify electrodes with MI below an MI threshold and excluding corresponding channels from the electrical readings.

20. A method for reducing artifacts in neural activity readings, the method including: acquiring raw neural activity readings recorded using multiple intracranial recording electrodes;
applying a band pass filter to the raw neural activity readings;
identifying an artifact epoch based on normalized mean amplitude across electrodes;
calculating mutual information (MI) across electrodes in the artifact epoch;
identifying electrodes with MI below an MI threshold and excluding corresponding channels from the raw neural activity readings;
performing, in incremental blocks, independent component analysis (ICA) on remaining neural activity readings;
pruning a first independent component (IC1) in each incremental block; and generating a report of the pruned neural activity readings.

21. The method of claim 20, further including:
calculating an artifact index (AI); and
excluding high-frequency oscillations, or action potentials occurring when the AI is above an AI threshold.

22. The method of claim 20, further including calculating an artifact index (AI), wherein the AI for a time series is calculated as
$$AI ( t ) = \frac{\vert {hfo}_{i} ( t ) - ic {1}_{i} ( t ) \vert}{Σ \vert {hfo}_{i} ( t ) \vert \text{/} T} * \frac{1}{I} {\sum }_{i}^{I} \frac{\vert {hfo}_{i} ( t ) - ic {1}_{i} ( t ) \vert}{Σ \vert {hfo}_{i} ( t ) \vert \text{/} T}$$
where hfoᵢ(t) is the band pass filtered EEG or LFP recording (i), and iclᵢ(t) is the band pass filtered EEG or LFP recording (i) after pruning the first independent component.

23. The method of claim 1, wherein pruning selected components in each iteration comprises:
calculating an inverse weight matrix for each of the consecutive iterations with raw and normalized matrix values;
applying a threshold to the inverse weight matrix calculated in each of the ICA iterations; and
removing an independent component of greatest order having raw and normalized matrix values that exceed the threshold in each of the consecutive iterations.

24. The system of claim 13, wherein pruning selected components includes the processor being configured to:
calculate an inverse weight matrix with raw and normalized matrix values;
apply a threshold to the inverse weight matrix; and
remove an independent component of greatest order having raw and normalized matrix values that exceed the threshold in each of the consecutive iterations.
